(19) United States
(12) Patent Application Publication
(10) Pub. No.: US 2011/0312891 A1
(43) Pub. Date: Dec. 22, 2011

(54) ENAMEL MATRIX DERIVATIVE FRACTION C

(75) Inventors: Stina Gestrelius, Lund (SE); Michel Dard, Liestal (CH); Ruzica Ranevski, Sissach (CH); S. Petter Lyngstadaas, Nesoddtangen (NO); Corinna Mauth, Laufenburg (CH)

(73) Assignee: Straumann Holding AG, Basel (CH)

(21) Appl. No.: 13/000,130

(22) PCT Filed: Jun. 29, 2009
(86) PCT No.: PCT/SE09/50832

§ 371 Date: Mar. 21, 2011

(30) Foreign Application Priority Data
Jun. 27, 2008 (SE) 080152703

(51) Int. Cl. A61K 38/17; A61P 19/08; C12N 5/0775; C07K 14/47; C12N 5/071

(52) U.S. Cl. 514/16.7; 530/324; 435/375

(57) ABSTRACT

Isolated active compound of a naturally occurring fraction of Enamel Matrix Derivatives (EMD), having at least one of each two N-terminal polypeptide fragments of amelogenin, which are at least 95% identical to an amino acid sequence as shown in SEQ. ID. No:1 and/or 2. The invention relates to the use of said isolated active compound of a fraction and/or of the fraction itself, and/or the at least one of each two polypeptide fragments for use as a medicament and/or for the manufacture of a pharmaceutical composition for a variety of different medical indications such as inducing and/or promoting cementogenesis, bone growth and/or binding between parts of living mineralised tissue, for bonding of a piece of living mineralised tissue to a bonding site on a piece of other living tissue, for endorsing binding between hard tissues, for inducing regeneration of dentin, and/or for filling a mineralized wound cavity and/or tissue defect following from a procedure and/or trauma.

### FIELD OF THE INVENTION

The present invention relates to an isolated active compound of a naturally occurring fraction of Enamel Matrix Derivatives (EMD), fraction C, which consists of at least one of each two polypeptide fragments of amelogenin as shown in SEQ. ID. No:1 and 2, produced naturally by alternate splicing and/or processing, or by either enzymatic or chemical cleavage of a natural length protein, or by synthesis of polypeptides in vitro or in vivo (e.g. recombinant DNA methods and/or cultivation of diploid cells). The present invention in particular relates to the use of said isolated faction C and/or said active compound of said fraction and/or the at least one of each two polypeptide fragments, for regulating activity, proliferation and/or differentiation of periodontal cells, for regulating osteoblast differentiation and/or proliferation, and/or for regulating mesenchymal stem cell proliferation and/or differentiation.

The present invention further relates to the use of said isolated active compound of a naturally occurring fraction of Enamel Matrix Derivatives (EMD), which consists of at least one of each two polypeptide fragments of amelogenin as shown in SEQ. ID. No:1 and 2, as a medicament. Furthermore, the present invention relates to the use of said isolated faction C and/or said active compound of said fraction and/or the at least one of each two polypeptide fragments, for the manufacture of a pharmaceutical composition for a variety of different medical indications such as inducing and/or promoting cementogenesis, bone growth and/or binding between parts of living mineralised tissue, for bonding of a piece of living mineralised tissue to a bonding site on a piece of other living tissue, for endorsing binding between hard tissues, for inducing regeneration of dentin, and/or for filling a mineralized wound cavity and/or tissue defect following from a procedure and/or trauma.

### BACKGROUND OF THE INVENTION

Enamel matrix proteins, present in the enamel matrix, are most well-known as precursors to enamel. Prior to cementum formation, enamel matrix proteins are deposited on the root surface at the apical end of the developing tooth-root. There is evidence that the deposited enamel matrix is the initiating factor for the formation of cementum. Again, the formation of cementum in itself is associated with the development of the periodontal ligament and the alveolar bone. Enamel matrix proteins can therefore promote periodontal regeneration through mimicking the natural attachment development in the tooth (Gestrelius S, Lyngstadaas S P, Hammarstrøm L. Emdogain—periodontal regeneration based on biomimicry. Clin Oral Invest 4:120-125 (2000). Isolated enamel matrix proteins are able to induce not only one but an orchestrated cascade of factors, naturally found in tissues developing adjacent to the enamel matrix. They mimic the natural environment of a developing tissue and thus mimic a natural stimulation for tissue regeneration, cell differentiation and/or maturation.

Enamel matrix derivative (EMD), in the form of a purified acid extract of proteins from pig enamel matrix, has previously been successfully employed to restore functional periodontal ligament, cementum and alveolar bone in patients with severe tooth attachment loss (Hammarstrøm et al., 1997, Journal of Clinical Periodontology 24, 658-668).

Furthermore, in studies on cultured periodontal ligament cells (PDL), it was shown that the attachment rate, growth and metabolism of these cells were significantly increased when EMD was present in the cultures. Also, cells exposed to EMD showed increased intracellular cAMP signalling and autocrine production of growth factors, when compared to controls. Epithelial cells on the other hand, although increasing cAMP signalling and growth factor secretion when EMD was present, were inhibited in both proliferation and growth (Lyngstadaas et al., 2001, Journal of Clinical Periodontology 28, 181-188).

Enamel matrix proteins and enamel matrix derivatives (EMD) have previously been described in the patent literature to be able to induce hard tissue formation (i.e. enamel formation, U.S. Pat. No. 4,672,032 (Slavkin)), endorse binding between hard tissues (EP-B-0 337 967 and EP-B-0 263 086), promote open wound healing, such as of skin and mucosa, have a beneficial effect on treatment of infections and inflammatory diseases (EPO 1, 1059934 and EPO II, 01201915.4), induce regeneration of dentin (WO 01/97834), promote the take of a graft (WO 00/53197), induce apoptosis in the treatment of neoplasms (WO 00/53196), regulate imbalance in an immune response to a systemic infection or inflammation (WO 03/024479), and to facilitate filling a wound cavity and/or tissue defect following from a procedure and/or trauma, such as a cytoreductive surgery (WO 02/080994).

The enamel matrix is composed of a number of proteins, such as amelogenins, enamelin, tuft protein, proteases, and albumin. Amelogenins, a major constituent of the enamel matrix, are a family of hydrophobic proteins derivable from a single gene by alternative splicing and controlled post secretory processing. They are highly conserved throughout vertebrate evolution and demonstrate a high overall level of sequence homology among all higher vertebrates examined (80%). In fact, the sequences of porcine and human amelogenin gene transcript differ only in 4% of the bases. Thus, enamel matrix proteins, although of porcine origin, are considered “self” when encountered in the human body and can promote dental regeneration in humans without triggering allergic responses or other undesirable reactions. Nonetheless, the plurality of structures identified in the different amelogenins studied, that will even occur in the same individual animal or human dentitions, clearly gives rise to speculations on the extreme specificity of the structures that work in concert in a “normal” amelogenesis. E.g., as shown by Li et al., 2003, a single base mutation in the x-chromosomal amelogenin gene, which results in a single proline to threonine change in the expressed human amelogenin, does give rise to amelogenesis imperfecta.

During cementogenesis in the developing tooth, amelogenin degrades into smaller pieces, and these pieces seem to interact differentially with the surrounding tissue and promote serial steps in the development of the periodontal system. As already described in Fincham et al, 1993, enamel contains a complex of amelogenin proteins which includes components ranging in size from 5-25 kDa. This is due to the expression and secretion of a family of amelogenins derivable from multiple mRNAs generated by differential splicing from one or two copies of the amelogenin gene, located on the X and Y chromosome. What is more, subsequent to secretion, these proteins appear further to undergo extensive proteolytic processing. Because of this extensive alternative splicing of the primary transcript and the following proteolytic processing of the secreted proteins, it has been difficult to assign functions to individual amelogenins. The pattern of splicing is unique for each amelogenin gene yet investigated, even when two copies of the gene are expressed in the same cell. Despite the high conservation of amelogenin sequences across species, diversity in the pattern of RNA splicing thus leads to significant differences in the number and character of amelogenin isoforms in the developing enamel matrix.

To date, two classes of amelogenin proteins have been described in the size of between 5-6 kDa, namely leucine-rich amelogenin polypeptide (LRAP) and tyrosine-rich amelogenin polypeptide (TRAP) (e.g. see Fincham et al., 1989). LRAP is translated from a shorter mRNA that has the coding regions from exons 4, 5 and part of 6 deleted during splicing. Due to its potential important regulatory effect as one of the processed fragments found of amelogenin, it was 2004 investigated by Boabaid et al, (Boabaid F., et al, J. Periodontol, Vol 75, No. 8, 2004) but was reported not to have any effect on cell proliferation in itself. What is more, it decreased the number of cementoblasts in cell culture, contrary to EMD which promotes cell proliferation of cementoblasts in vitro and full length amelogenin, which has no reported effect.

Two human tyrosine-rich amelogenin polypeptides (TRAPs) of approximately 5 kDa in size have prior been identified (see Fincham et al., 1989). These polypeptides were found to be of 42 (TRAP-2) and 44 (TRAP-1) amino acid residues in length; two forms of TRAP molecules, differing only by cleavage of a carboxy-terminal dipeptide, which were described to be a general feature of human and other mammalian enamel proteins, probably being derived by postsecretory cleavage from the primary extracellular amelogenin. No specific biological effect has so far been attributed to these polypeptides.

As an alternative to the rather blunt use of a complete cocktail of EMD, the separation of certain closely defined fractions and/or polypeptides or fragments of polypeptides from e.g. porcine tissues with specific biological activities, would allow a more refined use of enamel matrix protein, e.g. to induce specific steps during periodontal development, such as de novo bone formation or cementogenesis, or to mimic them in medical treatments. What is more, it might even be possible and cost efficient to synthesize single defined polypeptide sequences for use as separate and/or combined active components for inducing a specific desired effect.

The present invention for the first time identifies 2 naturally occurring porcine N-terminal amelogenin polypeptide fragments that together are shown to be able to induce osteogenic activity, such as proliferation of precursor cells and early differentiation of osteoblasts. The present invention also for the first time identifies which specific biological activity of EMD can be attributed to the complete fraction C, comprising said 2 naturally occurring N-terminal amelogenin polypeptide fragments.

### SUMMARY OF THE INVENTION

Amelogenin splice variants and proteolytic cleavage products are the main compounds isolated from EMD.

During cementogenesis in the developing tooth, amelogenin, as described above, due to alternative splicing of the primary transcript and the following proteolytic processing of the secreted proteins, degrades into smaller pieces (fragments and polypeptide fragments), and these pieces are hypothesised to interact differentially with the surrounding tissue and promote serial steps in the development of the periodontal system.

The present invention is based on the isolation of a specific fraction of porcine EMD, separated by High Pressure Liquid Chromatography (HPLC), hereafter termed fraction C, which is for the first time shown to comprise at least one of each of two polypeptides that were further separated and identified as shown in SEQ ID NO: 1 and SEQ ID NO: 2, and the finding that said fraction, as well as the isolated polypeptides in varying combinations with each other, execute specific biological functions that are closely related to, but not identical to the effects prior observed with EMD, or full-length amelogenin. The present invention thus for the first time successfully identifies said naturally occurring porcine N-terminal amelogenin polypeptide fragments that together are shown to be able to induce osteogenic activity.

The inventors further for the first time show cell culture studies indicating that the isolated fraction C of EMD and/or at least one of each of the 2 naturally occurring porcine N-terminal amelogenin polypeptide fragments can promote osteoblast proliferation and/or differentiation, as well as mesenchymal stem cell differentiation, as well as periodontal cell activation. The present inventors convincingly demonstrate that the main fraction in itself and the combined at least 2 polypeptide fragments have different biological activities and can be used separately, as well as in combination.

Thus, the present invention relates to the combined isolated naturally occurring porcine N-terminal amelogenin polypeptide fragments as shown in SEQ ID NO: 1 and SEQ ID NO: 2 and to a pharmaceutical preparation comprising at least one of each of said 2 naturally occurring porcine N-terminal amelogenin polypeptide fragments, as well as to said combined isolated naturally occurring porcine N-terminal amelogenin polypeptide fragments as shown in SEQ ID NO: 1 and SEQ ID NO: 2 for use in medicicine.

The present invention furthermore relates to the use of said certain naturally occurring fraction C of enamel matrix derivatives, or to the at least one of each two polypeptide fragments of amelogenin as shown in SEQ. ID. No:1 and 2, or to a pharmaceutical preparation which consist of at least one of each two polypeptide fragments of amelogenin as shown in SEQ. ID. No:1 and 2, or of fraction C, produced naturally by alternate splicing or processing, or by either enzymatic or chemical cleavage of a natural length protein, or by synthesis of polypeptides in vitro or in vivo (e.g. recombinant DNA methods and/or cultivation of diploid cells) for activating and/or regulating activity of periodontal cells, regulating osteoblast differentiation and/or proliferation, and/or regulating mesenchymal stem cell proliferation and/or differentiation.

Consequently, the present invention also relates to the use of said fraction C or at least one of each of the polypeptide fragments of said naturally occurring fraction of enamel matrix derivatives, produced naturally by alternate splicing and/or processing, or by either enzymatic or chemical cleavage of a natural length protein, or by synthesis of polypeptides in vitro or in vivo (e.g. recombinant DNA methods and/or cultivation of diploid cells) to exert a specific action, such as regulating activity of periodontal cells, regulating osteoblast differentiation and/or proliferation, and/or regulating mesenchymal stem cell proliferation and/or differentiation.

As e.g. summarised in table 2, experiments conducted with primary cell cultures of human osteoblasts clearly showed that the isolated fraction of enamel proteins, referred to herein as fraction C, exerts a biologically significant effect on a broad variety of genes, well-known in the field of the art to be involved in apoptosis, cell adhesion, cell-cell signalling, transcription, signal transduction, and/or cell proliferation. What is more, the documented effect was closely related to but not identical to the effect seen with EMD.

One embodiment of the present invention thus relates to the use of an isolated fraction of enamel matrix proteins, fraction C, which comprises at least one of each two polypeptide fragments of amelogenin as shown in SEQ. ID. No:1 and 2, or to a pharmaceutical preparation which comprises at least one of each two polypeptide fragments of amelogenin as shown in SEQ. ID. No:1 and 2, produced naturally by alternate splicing or processing, or by either enzymatic or chemical cleavage of a natural length protein, or by synthesis of polypeptides in vitro or in vivo (e.g. recombinant DNA methods and/or cultivation of diploid cells) for activating and/or regulating activity of periodontal cells, and/or for regulating osteoblast differentiation and/or proliferation, for regulating mesenchymal stem cell proliferation and/or differentiation, e.g. into osteoblast cells.

In another, equally preferred embodiment, the present invention relates to the use of at least one of each two polypeptide fragments of amelogenin as shown in SEQ. ID. No:1 and 2, for activating and/or regulating activity of periodontal cells, and/or for regulating osteoblast differentiation and/or proliferation, for regulating mesenchymal stem cell proliferation and/or differentiation, e.g. into osteoblast cells, and/or for inducing an immune response.

Enamel matrix proteins are known to activate certain signalling pathways, promote proliferation, and induce differentiation in periodontal cells and to stimulate non-periodontal fibroblast cell growth and differentiation, whereas epithelial cell growth and/or differentiation is not stimulated by the presence of enamel matrix proteins.

As shown prior by the present inventors, the increased attachment rate of non-periodontal fibroblast cells that grow on active enamel substances demonstrates that an enamel protein based matrix mimics an extracellular matrix. This mimicry facilitates rapid attachment of these cells. The observed rise in growth rate and metabolism in these fibroblast cells, growing on active enamel substances, further proves that the fraction and/or polypeptide fragments of an active enamel substances provides an extracellular matrix that stimulates periodontal cells to speed up their metabolism. Also, a rise in growth rate is reflected in the increase of DNA synthesis, indicating that cell proliferation is up-regulated in these cultures. Furthermore, since the increase in utilisation of [35S]-methionine in these fibroblast cells exceeds the rise in growth rate, some of the added metabolic activity also reflects a boosted anabolism and/or secretion of extracellular proteins. Due to the herein documented effects of fraction C on the activity of periodontal cells, a similar effect is presently envisioned to be exerted by it, as well as by at least one of each two polypeptide fragments of amelogenin as shown in SEQ. ID. No:1 and 2.

In particular, the presently presented experimental findings clearly show that fraction C is the component in EMD that acts on osteoblasts and which has both osteogenic and cementogenic potential, thus suggesting that it is the active component of EMD and amelogenin with respect to bone and cementum regeneration. In particular again, the inventors found that fraction C, as well as the combined two polypeptide fragments of amelogenin as shown in SEQ. ID. No:1 and 2, were upregulating activity of periodontal cells, and/or upregulating very early osteoblast differentiation and/or proliferation markers, as well as upregulating mesenchymal stem cell proliferation and/or reducing and/or inhibiting differentiation of mesenchymal stem cells. Especially, the effect of the combined two polypeptide fragments of amelogenin as shown in SEQ. ID. No:1 and 2, was more strongly detected as a strong inducer or fascilitator of proliferation of osteoblast or mesenchymal cells, whereas the effect on early differentiation, measured by the expression of marker genes in these cells, was rather negative.

Thus, without wishing to limit the present invention to a specific scientific theory, it is herein envisioned that fraction C is the earliest active fraction of EMD, comprising an assertion of components of EMD that will induce an instant proliferating stimuli into the surrounding tissue, at an early stage prioritising the amassment of undifferentiated cells over the specification of them. Especially, the two combined polypeptide fragments of amelogenin as shown in SEQ. ID. No:1 and 2, clearly demonstrate a strong biological effect on the induction of proliferation in osteoblast like precursor cells as well as in PDL cells, and could even be shown to reduce the level of later differentiation markers. Fraction C as a complete fraction does, in contrast to the before mentioned inhibiting effects of the combined peptides, not only stimulate the proliferation of pluripotent and/or omnipotent cells, but could be shown to be able to induce very early markers for osteogenic and chondrogenic differentiation of said cells.

An “osteoblast” is an uninucleated cell that synthesize both collagenous and noncollagenous bone proteins (the organic matrix, osteoid). They are responsible for mineralization and are derivable from a multipotent mesenchymal cell. The osteoblast is generally considered to differentiate through a precursor cell, the preosteoblast.

As shown in the experimental section, in example 3 and 4, fraction C increases osteoblast differentiation and stimulates osteogenesis in cultured osteoblasts. What is more, example 5 demonstrates clearly that isolated combined at least one of each two polypeptide fragments of amelogenin as shown in SEQ. ID. No:1 and 2 is more potent than fraction C in stimulating early osteoblast differentiation, whereas fraction C seems to be a more potent inducer of osteocalcin expression at later stages.

The cells which form part of the periodontal ligament (PDL), are mainly fibroblasts. In the PDL, the fibroblasts are characterized by an ability to achieve an exceptionally high rate of turnover of the extracellular compartment, in particular, collagen. Ligament fibroblasts are aligned along the general direction of the fiber bundles and with extensive processes that wrap around the fiber bundles. Also epithelial cells and undifferentiated mesenchymal cells are constituents of the PDL.

The term “periodontal cells”, in the present context, refers to cells such as periodontal ligament cells (PDL), gingival cells, epithelial cells and/or bone cells, but is not limited thereto.

“Differentiation” of a cell, refers to a process by which a cell undergoes a change to an overtly specialized cell type. Such a cell may be a stem cell differentiating into other specialized cell types during embryogenesis or later stages of development, or any other cell receiving instructions to do so. A typical example for differentiation would in the present context e.g. be the differentiation of mesenchymal stem cells into osteoblasts.

“Proliferation” of a cell refers to a stage wherein the cell actively is growing and dividing to generate a cell population of a greater size. Such proliferation may be stimulated by external stimuli, such as growth factors etc.

“Mesenchyme” refers to an immature, unspecialized form of connective tissue in animals, consisting of cells embedded in a tenuous extracellular matrix. Embryonic connective tissue derivable from mesoderm, is named mesenchyme. “Mesenchymal stem cells” are undifferentiated mesenchyme cells, such as bone marrow cells. In a presently preferred embodiment, said mesenchymal stem cells are differentiated into e.g. osteoblasts, osteoclasts, or any other bone cell.

In close analogy to the documented cellular effects seen in the experiments performed and documented in the experimental section above, another aspect of the present invention relates to the use of isolated fraction C and/or the use of an isolated fraction of enamel matrix proteins, which consists of at least one of each two polypeptide fragments of amelogenin as shown in SEQ. ID. No:1 and 2, or to a pharmaceutical preparation, which comprises at least one of each two polypeptide fragments of amelogenin as shown in SEQ. ID. No:1 and 2, produced naturally by alternate splicing or processing, or by either enzymatic or chemical cleavage of a natural length protein, or by synthesis of polypeptides in vitro or in vivo (e.g. recombinant DNA methods and/or cultivation of diploid cells), or to at least one of each two isolated polypeptide fragments of amelogenin as shown in SEQ. ID. No:1 and 2, for the manufacture of a pharmaceutical composition for inducing mineralization in hard tissue.

Yet another aspect of the present invention relates to the use of isolated fraction C and/or the use of an isolated fraction of enamel matrix proteins, which consists of at least one of each two polypeptide fragments of amelogenin as shown in SEQ. ID. No:1 and 2, or to a pharmaceutical preparation, which comprises at least one of each two polypeptide fragments of amelogenin as shown in SEQ. ID. No:1 and 2, produced naturally by alternate splicing or processing, or by either enzymatic or chemical cleavage of a natural length protein, or by synthesis of polypeptides in vitro or in vivo (e.g. recombinant DNA methods and/or cultivation of diploid cells), or to at least one of each two isolated polypeptide fragments of amelogenin as shown in SEQ. ID. No:1 and 2, produced naturally by alternate splicing or processing, or by either enzymatic or chemical cleavage of a natural length protein, or by synthesis of polypeptides in vitro or in vivo (e.g. recombinant DNA methods and/or cultivation of diploid cells), for the manufacture of a pharmaceutical composition for inducing bone re-growth and/or de novo growth, for inducing hard tissue formation, for endorsing binding between hard tissues, and/or for inducing regeneration of dentin.

A presently preferred embodiment of the invention thus relates to an isolated fraction of enamel matrix proteins, which consists of at least one of each two polypeptide fragments of amelogenin as shown in SEQ. ID. No:1 and 2, or a pharmaceutical preparation, which comprises at least one of each two polypeptide fragments of amelogenin as shown in SEQ. ID. No:1 and 2, produced naturally by alternate splicing or processing, or by either enzymatic or chemical cleavage of a natural length protein, or by synthesis of polypeptides in vitro or in vivo (e.g. recombinant DNA methods and/or cultivation of diploid cells), or to at least one of each two isolated polypeptide fragments of amelogenin as shown in SEQ. ID. No:1 and 2, produced naturally by alternate splicing or processing, or by either enzymatic or chemical cleavage of a natural length protein, or by synthesis of polypeptides in vitro or in vivo (e.g. recombinant DNA methods and/or cultivation of diploid cells), for use as a medicament.

Enamel matrix proteins are able to induce dentin formation in dental pulp cells. Accordingly, a similar effect is presently envisioned to be exerted by the present fraction C, which comprises at least one of each two polypeptide fragments of amelogenin as shown in SEQ. ID. No:1 and 2, or by an isolated fraction of enamel matrix proteins, which consists of at least one of each two polypeptide fragments of amelogenin as shown in SEQ. ID. No:1 and 2, or a pharmaceutical preparation, which comprises at least one of each two polypeptide fragments of amelogenin as shown in SEQ. ID. No:1 and 2, produced naturally by alternate splicing or processing, or by either enzymatic or chemical cleavage of a natural length protein, or by synthesis of polypeptides in vitro or in vivo (e.g. recombinant DNA methods and/or cultivation of diploid cells), or to at least one of each two isolated polypeptide fragments of amelogenin as shown in SEQ. ID. No:1 and 2, produced naturally by alternate splicing or processing, or by either enzymatic or chemical cleavage of a natural length protein, or by synthesis of polypeptides in vitro or in vivo (e.g. recombinant DNA methods and/or cultivation of diploid cells), on the formation or regeneration of dentin following dental procedures involving exposure of vital dental pulp tissue.

In another aspect, the invention relates to a method of promoting the formation or regeneration of dentin following dental procedures involving exposure of vital dental pulp tissue, the method comprising applying an effective amount of an isolated fraction C and/or an isolated fraction of enamel matrix proteins, which consists of at least one of each two polypeptide fragments of amelogenin as shown in SEQ. ID. No:1 and 2, or a pharmaceutical preparation, which comprises at least one of each two polypeptide fragments of amelogenin as shown in SEQ. ID. No:1 and 2, produced naturally by alternate splicing or processing, or by either enzymatic or chemical cleavage of a natural length protein, or by synthesis of polypeptides in vitro or in vivo (e.g. recombinant DNA methods and/or cultivation of diploid cells), or to at least one of each two isolated polypeptide fragments of amelogenin as shown in SEQ. ID. No:1 and 2, produced naturally by alternate splicing or processing, or by either enzymatic or chemical cleavage of a natural length protein, or by synthesis of polypeptides in vitro or in vivo (e.g. recombinant DNA methods and/or cultivation of diploid cells), on exposed vital dental pulp tissue after dental procedures.

The present invention relates to the use of isolated fraction C and/or the use of an isolated fraction of enamel matrix proteins, which consists of at least one of each two polypeptide fragments of amelogenin as shown in SEQ. ID. No:1 and 2, or a pharmaceutical preparation, which comprises at least one of each two polypeptide fragments of amelogenin as shown in SEQ. ID. No:1 and 2, produced naturally by alternate splicing or processing, or by either enzymatic or chemical cleavage of a natural length protein, or by synthesis of polypeptides in vitro or in vivo (e.g. recombinant DNA methods and/or cultivation of diploid cells), or to at least one of each two isolated polypeptide fragments of amelogenin as shown in SEQ. ID. No:1 and 2, produced naturally by alternate splicing or processing, or by either enzymatic or chemical cleavage of a natural length protein, or by synthesis of polypeptides in vitro or in vivo (e.g. recombinant DNA methods and/or cultivation of diploid cells), for application on medical implants or devices. The invention also relates to medical implants or devices onto which an isolated fraction C and/or an isolated fraction of enamel matrix proteins, which consists of at least one of each two polypeptide fragments of amelogenin as shown in SEQ. ID. No:1 and 2, or a pharmaceutical preparation, which comprises at least one of each two polypeptide fragments of amelogenin as shown in SEQ. ID. No:1 and 2, produced naturally by alternate splicing or processing, or by either enzymatic or chemical cleavage of a natural length protein, or by synthesis of polypeptides in vitro or in vivo (e.g. recombinant DNA methods and/or cultivation of diploid cells), or to at least one of each two isolated polypeptide fragments of amelogenin as shown in SEQ. ID. No:1 and 2, produced naturally by alternate splicing or processing, or by either enzymatic or chemical cleavage of a natural length protein, or by synthesis of polypeptides in vitro or in vivo (e.g. recombinant DNA methods and/or cultivation of diploid cells), has been applied.

According to the present invention, the implant or device may be any implant or device intended for use in the human or animal body, in particular in the dental area, gastrointestinal tract, urethra, bladder, pulmonary cavity, lungs, trachea, larynx, oesophagus, joints, bone, skull, ears, sinuses, veins, arteries or abdominal cavity. The implant can a bone substitute material, such as ceramic and or plaster.

The implant or device may be used for fixation of complicated fractures, e.g. of the neck, legs or arms, or skull fractures, thus the implant or device may be a pin or screw or bone substitute material, conventionally used to immobilise (fix) fragments of fractured bone. Such pins or screws typically comprise a portion that penetrates the skin of the patient at or near the site of the fracture. Pins and screws for this purpose may conventionally be prepared from a metal such as titanium or steel, and may optionally be coated with a polymeric material which may typically be biodegradable or stabilised to facilitate soft tissue closure and sealing. Furthermore, an implant may be an electrical conductor such as one used in, e.g., pacemakers, brain implants or biosensors. The implant may also be an artificial tooth or a dental prothesis, such as a screw and/or an abutment.

Before application on an implant or device, the present isolated fraction C and/or the isolated fraction of enamel matrix proteins, which consists of at least one of each two polypeptide fragments of amelogenin as shown in SEQ. ID. No:1 and 2, or a pharmaceutical preparation, which comprises at least one of each two polypeptide fragments of amelogenin as shown in SEQ. ID. No:1 and 2, produced naturally by alternate splicing or processing, or by either enzymatic or chemical cleavage of a natural length protein, or by synthesis of polypeptides in vitro or in vivo (e.g. recombinant DNA methods and/or cultivation of diploid cells), or to at least one of each two isolated polypeptide fragments of amelogenin as shown in SEQ. ID. No:1 and 2, produced naturally by alternate splicing or processing, or by either enzymatic or chemical cleavage of a natural length protein, or by synthesis of polypeptides in vitro or in vivo (e.g. recombinant DNA methods and/or cultivation of diploid cells), may be admixed with other ingredients, e.g. pharmaceutically acceptable excipients to constitute a pharmaceutical composition, as discussed below, and coated onto the surface of the implant or device, e.g. by dipping the relevant portion of the implant or device in a solution or dispersion of the active enamel substance or by spraying a solution or dispersion of the active enamel substance onto the relevant surface of the implant or device followed, in both cases, by drying. On application, the fraction C, pharmaceutical preparation, and/or polypeptide fragment combination is adsorbed to the surface of the implant or device and may be fixed thereon by means of conventional fixatives such as formaldehyde, glutaraldehyde or ethanol. Alternatively, the fraction C, pharmaceutical preparation, and/or polypeptide fragment combination may be applied on the relevant surface of the implant or device by cross-linking said fraction and/or polypeptide fragment of an active enamel substance, to a polymer component of the implant or device, e.g. by UV radiation or chemical treatment in a manner known per se, or by covalently binding the fraction and/or polypeptide fragment to a suitable functional group of a polymeric component present on the surface of the implant or device.

The amount of fraction C and/or polypeptide fragment applied on the appropriate surface of the implant or device will normally result in an amount of total protein per cm² area of the implant or device corresponding to from about 0.005 mg/cm² to about 20 mg/cm² such as from about 0.01 mg/cm² to about 15 mg/cm².

In accordance with the present invention, application of the fraction C, pharmaceutical preparation, and/or polypeptide fragment combination according to the present invention on a surface of an implant or device for the present purpose may optionally be combined with application of other types of suitable biologically active substances, e.g. antimicrobial agents such as antibacterial or antifungal agents, or application of bacteriostatic agents or disinfectants for the prevention or treatment of microbial infections at the site where the implant or device is in contact with epithelial tissue.

“Soft tissues”, (i.e. non-mineralised tissues), can in the present context be used interchangeably with gingival tissue, and may be defined as collagen or epithelium containing tissues, including skin and mucosa, muscle, blood and lymph vessels, nerve tissues, glands, tendons, eyes and cartilage. In general the fraction and/or polypeptide fragments of the present invention can be used to promote healing or for manufacturing a pharmaceutical composition for promoting healing of a wound not only in skin and mucosa, but in any gingival tissue of the patient in need thereof.

The term “hard-tissue formation” in “mineralised tissue” may be summarised as the production by cells of an organic matrix capable of accepting mineral, with the activity of the enzyme alkaline phosphatase and a good blood supply prerequisites.

In accordance with the present invention, the presently isolated fraction C and/or the isolated fraction of enamel matrix proteins, which consists of at least one of each two polypeptide fragments of amelogenin as shown in SEQ. ID. No:1 and 2, or the pharmaceutical preparation, which comprises at least one of each two polypeptide fragments of amelogenin as shown in SEQ. ID. No:1 and 2, produced naturally by alternate splicing or processing, or by either enzymatic or chemical cleavage of a natural length protein, or by synthesis of polypeptides in vitro or in vivo (e.g. recombinant DNA methods and/or cultivation of diploid cells), or to at least one of each two isolated polypeptide fragments of amelogenin as shown in SEQ. ID. No:1 and 2, produced naturally by alternate splicing or processing, or by either enzymatic or chemical cleavage of a natural length protein, or by synthesis of polypeptides in vitro or in vivo (e.g. recombinant DNA methods and/or cultivation of diploid cells) will typically originate from porcine.

None withstanding, in light of the well known fact that amelogenin is an evolutionary very conservative protein, and the homology in between the species is documented to be high, it is presently envisioned that analogous sequences might be found in rat, human, or mouse enamel matrix proteins, e.g. in amelogenin, which exert similar biological effects, e.g. possess osteogenic activity. The present invention consequently also encompasses analogue sequences to porcine amelogenin fragments as disclosed in ID. SEQ. NO.: 1 and 2, which are at least 95% identical to at least one of the amino acid sequences shown in ID. SEQ. NO: 1 and 2, such as at least 95%, 96%, 97%, 98%, 99%, or 99.5% identical, and which show analogue biological activity. In the present invention, such analogue polypeptides are envisioned to be usable for producing medicaments and/or pharmaceutical and/or cosmetical compositions for inducing mineralization in hard tissue and/or for inducing bone growth and/or bone regrowth.

In the present invention, a polypeptide fragment selected from the group consisting of polypeptide fragments which are at least 95% identical to at least one of the amino acid sequences shown in ID SEQ NO: 1 and 2, such as SEQ ID NO: 1 and/or SEQ ID NO: 2, can be selected from a polypeptide fragment isolated from mammalian tissue, a purified recombinant polypeptide fragment, or a polypeptide fragment which is synthetically manufactured. As is well known in the art, a recombinantly produced polypeptide will differ slightly from the endogenous template protein, especially when it is produced in a prokaryotic system. The present invention encloses recombinantly produced polypeptide fragments which are at least 95% identical to at least one of the amino acid sequences shown in ID. SEQ. NO: 1 and 2, such as at least 95%, 96%, 97%, 98%, 99%, or 99.5% identical, and which show analogue biological activity.

A synthetically manufactured polypeptide on the other hand, as is well known in the art, can of course be designed to carry a diversity of chemical permutations that will not hinder and/or effect its original biological activity, e.g. its chondrogenic and/or its osteogenic activity. Consequently, the present invention encloses also synthetically permutated polypeptide fragments which are at least 95% identical to at least one of the amino acid sequences shown in ID. SEQ. NO: 1 and 2, such as at least 95%, 96%, 97%, 98%, 99%, or 99.5% identical, and which show analogue biological activity.

Additionally, any conservative variant of the sequence of a polypeptide fragment which is at least 95% identical to at least one of the amino acid sequences shown in ID. SEQ. NO.: 1 and 2, such as at least 95%, 96%, 97%, 98%, 99%, or 99.5% identical, and which shows analogue biological activity, is by virtue of its functional relationship to said sequences considered to be inside the scope of the present invention.

A conservative variant of a sequence is in the present context defined as an amino acid sequence which is conserved at least 95%, 96%, 97%, 98%, or 99%, when comparing variants of the same amino acid sequence between different species. The degree of conservation of a variant can, as is well known in the field, be calculated according to its derivation of PAM (see Dayhoff, Schwartz, and Orcutt (1978) Atlas Protein Seq. Struc. 5:345-352), or based on comparisons of Blocks of sequences derived from the Blocks database as described by Henikoff and Henikoff (1992) Proc Natl Acad Sci USA 89(22):10915-9.

Conservative substitutions may be made, for example according to table 1 below. Amino acids in the same block in the second column and preferably in the same line in the third column may be substituted for each other:

TABLE 1

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

Such replacements may also be made by unnatural amino acids include; alpha* and alpha-disubstituted* amino acids, N-alkyl amino acids*, lactic acid*, halide derivatives of natural amino acids such as trifluorotyrosine*, p-Cl-phenylalanine*, p-Br-phenylalanine*, p-I-phenylalanine*, L-allyl-glycine*, β-alanine*, L-a-amino butyric acid*, L-g-amino butyric acid*, L-a-amino isobutyric acid*, L-e-amino caproic acid#, 7-amino heptanoic acid*, L-methionine sulfone#*, L-norleucine*, L-norvaline*, p-nitro-L-phenylalanine*, L-hydroxyproline#, L-thioproline*, methyl derivatives of phenylalanine (Phe) such as 4-methyl-Phe*, pentamethyl-Phe*, L-Phe (4-amino)#, L-Tyr (methyl)*, L-Phe (4-isopropyl)*, L-Tic (1,2,3,4-tetrahydroisoquinoline-3-carboxyl acid)*, L-diaminopropionic acid # and L-Phe (4-benzyl)*. The notation * is herein utilised to indicate the hydrophobic nature of the derivative whereas # is utilised to indicate the hydrophilic nature of the derivative, #* indicates amphipathic characteristics.

Variant amino acid sequences may include suitable spacer groups that may be inserted between any two amino acid residues of the sequence including alkyl groups such as methyl, ethyl or propyl groups in addition to amino acid spacers such as glycine or b-alanine residues. A further form of variation, involves the presence of one or more amino acid residues in peptoid form, which will be well understood by those skilled in the art. For the avoidance of doubt, “the peptoid form” is used to refer to variant amino acid residues wherein the a-carbon substituent group is on the residue's nitrogen atom rather than the a-carbon. Processes for preparing peptides in the peptoid form are known in the art, see for example, Simon I U et al., PNAS (1992) 89(20), 9367-9371 and Norwell D C, Trends Biotechnol. (1995) 13(4), 132-134.

Polypeptides of the invention may be in a substantially isolated form. It will be understood that the peptide may be mixed with carriers or diluents, which will not interfere with the intended purpose of the peptide and still be regarded as substantially isolated. A peptide of the invention may also be in a substantially purified form, in which case it will generally comprise the peptide or a fragment thereof in a preparation in which more than 90%, e.g. 95%, 98% or 99% of the protein in the preparation is a peptide of the invention.

In the present context, a polypeptide fragment as shown in SEQ ID NO: 1 and SEQ ID NO: 2 does include such a recombinantly produced, or chemically manufactured polypeptide which is at least 95%, such as 95%, 96%, 97%, 98%, 99% or 99.9% identical with at least one of the sequences shown in SEQ ID NO: 1 and SEQ ID NO: 2, and which exerts the same biological effect, e.g. possesses chondrogenic, proliferative and/or osteogenic activity.

EMD is processed as described in the experimental section. The 2 separate peaks from fraction C (termed C1RP and C2RP) have been separated and the amino acid sequences of them have been determined as:

C1RP:
(SEQ.ID.NO: 1)
MPLPPHPGHPGYINFSYEVLTPLKWYQNMIRHP-YTSYGYEPMG
(43AA form)
C2RP:
(SEQ.ID.NO: 2)
MPLPPHPGHPGYINFSYEVLTPLKWYQNMIRHP-YTSYGYEPMGGW
(45AA form)

Although C1RP (SEQ. ID. NO:1) is predominantly described as being only 43 amino acids long, it can be extended to include at the most 45 amino acids, the C-terminally added amino acids being GW and be reduced to include at the least 41 amino acids, see SEQ. ID. NOs: 1 and 3-11. SEQ. ID. NOs: 1 and 3-11 are in the present context interchangeable with each other.

Although C2RP (SEQ. ID. NO:2) is predominantly described as being 45 amino acids long, it can be reduced to include at the least 42 amino acids, see SEQ. ID. NOs: 2 and 12-19. SEQ. ID. NOs: 2 and 12-19 are in the present context interchangeable with each other.

In the present context it is specifically noteworthy that the above identified polypeptides are not necessarily the same, but that they can differ in positions 1, 6, 7, and 16 and in their lengths. Thus, the molecular weights of the separate sequences are slightly but consistently different, even when both C1RP and C2RP consist of 45 amino acids each (m/z C1RP: 5160.39 Da, 5080.42, 4931.29; m/z C2RP: 5403.49 Da, 5175.40 Da). In another, equally preferred embodiment, 2 main peaks are identified for the polypeptides as m/z C1RP: 5160.8 Da, m/z C2RP: 5404.1 Da.

While not wishing to limit the present invention to one scientific theory, it is envisioned that the amelogenin polypeptides as shown in SEQ. ID. NO:1 and 2 form polymers with each other and/or aggregate and are biologically active as polymers, dimmers and/or aggregates. The two main peaks show many aromatic and ionic amino acids in the sequences, indicating that they have a strong interaction between them. Typically, the interaction form is n-stacking and ionic and hydrogen bonding interaction. These phenomena are shown with a UV melting curve and a MALDI measurement.

The present invention comprises the following slight variations from sequence C1RP and/or C2RP (SEQ. ID. NO:1 and 2):

### C1RP

### SEQ ID NO:3:

45 aa terminating with W, position 16 may be S or E, S may be phosphorylated in position 16

MPLPPHPGHPGYINF(S or E)YEVLTPLKWYQNMIRHPYTSYGYEPM
GGW

### SEQ ID NO:4:

45 aa terminating with W, position 16 (S) may be phosphorylated and position 42 (M) may be oxidized

MPLPPHPGHPGYINFSYEVLTPLKWYQNMIRHPYTSYGYEPMGGW

### SEQ ID NO:5:

44 aa terminating with G, position 16 (S) may be phosphorylated and position 42 (M) may be oxidized

MPLPPHPGHPGYINFSYEVLTPLKWYQNMIRHPYTSYGYEPMGG

### SEQ ID NO: 6

43 aa terminating with G, position 16 (S) may be phosphorylated and position 42 (M) may be oxidized

MPLPPHPGHPGYINFSYEVLTPLKWYQNMIRHPYTSYGYEPMG

### SEQ ID NO:7:

44 aa terminating with W, position 16 (S) may be phosphorylated and position 42 (M) is absent

MPLPPHPGHPGYINFSYEVLTPLKWYQNMIRHPYTSYGYEPGGW

### SEQ ID NO:8:

43 aa terminating with W, position 16 (S) may be phosphorylated and positions 42 (M) and 41 (P) are absent

MPLPPHPGHPGYINFSYEVLTPLKWYQNMIRHPYTSYGYEGGW

### SEQ ID NO:9:

43 aa terminating with G, position 16 (S) may be phosphorylated and position 42 (M) is absent

MPLPPHPGHPGYINFSYEVLTPLKWYQNMIRHPYTSYGYEPGG

### SEQ ID NO:10:

42 aa terminating with G, position 16 (5) may be phosphorylated and positions 42 (M) and 41 (P) are absent

MPLPPHPGHPGYINFSYEVLTPLKWYQNMIRHPYTSYGYEGG

### SEQ ID NO:11:

41 aa terminating with G, position 16 (S) may be phosphorylated and positions 42 (M) and 41 (P) are absent

MPLPPHPGHPGYINFSYEVLTPLKWYQNMIRHPYTSYGYEG

### C2RP

### SEQ ID NO:12:

45 aa terminating with W, position 16 may be S or E, S may be phosphorylated in position 16

MPLPPHPGHPGYINF(S or E)YEVLTPLKWYQNMIRHPYTSYGYEPM
GGW

### SEQ ID NO:13:

45 aa terminating with W, position 16 (5) may be phosphorylated and position 42 (M) may be oxidized

MPLPPHPGHPGYINFSYEVLTPLKWYQNMIRHPYTSYGYEPMGGW

### SEQ ID NO:14:

44 aa terminating with G, position 16 (5) may be phosphorylated and position 42 (M) may be oxidized

MPLPPHPGHPGYINFSYEVLTPLKWYQNMIRHPYTSYGYEPMGG

### SEQ ID NO:15:

43 aa terminating with G, position 16 (S) may be phosphorylated and position 42 (M) may be oxidized

MPLPPHPGHPGYINFSYEVLTPLKWYQNMIRHPYTSYGYEPMG

### SEQ ID NO:16:

44 aa terminating with W, position 16 (S) may be phosphorylated and position 42 (M) is absent

MPLPPHPGHPGYINFSYEVLTPLKWYQNMIRHPYTSYGYEPGGW

### SEQ ID NO:17:

43 aa terminating with W, position 16 (S) may be phosphorylated and positions 42 (M) and 41 (P) are absent

MPLPPHPGHPGYINFSYEVLTPLKWYQNMIRHPYTSYGYEGGW

### SEQ ID NO:18:

43 aa terminating with G, position 16 (S) may be phosphorylated and position 42 (M) is absent

MPLPPHPGHPGYINFSYEVLTPLKWYQNMIRHPYTSYGYEPGG

### SEQ ID NO:19:

42 aa terminating with G, position 16 (S) may be phosphorylated and positions 42 (M) and 41 (P) are absent

MPLPPHPGHPGYINFSYEVLTPLKWYQNMIRHPYTSYGYEGG

In an alternative embodiment, any of the identified sequences above can further be oxidated in at least one of the positions 1 and 42. In yet another embodiment, any of the identified sequences above can also be glycolysied in at least one of the positions 36 (S), 21 (T), 35 (T), 14 (N) and 28 (N).

### Active Enamel Substances

As used herein, “enamel matrix” means a precursor to enamel and may be obtained from any relevant natural source, i.e. a mammal in which teeth are under development. A suitable source is developing teeth from slaughtered animals such as, e.g., calves, pigs or lambs. Another source is e.g. fish skin. In the present context, the term “an active enamel substance” is used to encompass enamel matrix derivatives and/or enamel matrix proteins nondiscriminant of their source.

Enamel matrix can be prepared from developing teeth as described previously (EP-B-0 337 967 and EP-B-0 263 086). The enamel matrix is scraped off and enamel matrix derivatives are prepared, e.g. by extraction with aqueous solution such as a buffer, a dilute acid or base or a water/solvent mixture, followed by size exclusion, desalting or other purification steps, alternatively followed by freeze-drying. Enzymes may alternatively be deactivated by treatment with heat or solvents, in which case the derivatives may be stored in liquid form without freeze-drying.

As an alternative source of the enamel matrix derivatives or proteins one may also use generally applicable synthetic routes, well known to a person skilled in the art, or use cultivated eukaryotic and/or prokaryotic cells modified by DNA-techniques. The enamel matrix proteins may thus be of recombinant origin and alternatively genetically and/or chemically modified (see, e.g., Sambrook, J. et al.: Molecular Cloning, Cold Spring Harbor Laboratory Press, 1989).

In the present context, enamel matrix derivatives are derivatives of enamel matrix which include one or several enamel matrix proteins or parts or fragments of such proteins, produced naturally by alternate splicing or processing, or by either enzymatic or chemical cleavage of a natural length protein, or by synthesis of polypeptides in vitro or in vivo (e.g. recombinant DNA methods and/or cultivation of diploid cells). Enamel matrix protein derivatives also include enamel matrix related polypeptides or proteins. The polypeptides or proteins may be bound to a suitable biodegradable carrier molecule, such as polyamine acids or polysaccharides, or combinations thereof. Furthermore, the term enamel matrix derivatives also encompass synthetic analogous substances.

Proteins are biological macromolecules constituted by amino acid residues linked together by peptide bonds. Proteins, as linear polymers of amino acids, are also called polypeptides. Typically, proteins have 50-800 amino acid residues and hence have molecular weights in the range of from about 6,000 to about several hundred thousand Dalton or more. Small proteins are called peptides, oligopeptides or polypeptides. In the context of the present invention, a “polypeptide fragment” for use in accordance with the present invention, refers to a polypeptide which may be, but is not limited to, being 1-50 amino acids in length, such as 5, 10, 15, 20, 25, 30, 35, 40, 41, 42, 43, 44, 45, 46, 47, 47, 48, 49 or 50 amino acids. Such polypeptides may also be longer than 50 amino acids.

Enamel matrix proteins are proteins that normally are present in enamel matrix, i.e. the precursor for enamel (Ten Cate: Oral Histology, 1994; Robinson: Eur. J. Oral Science, January 1998, 106 Suppl. 1:282-91), or proteins which can be obtained by cleavage of such proteins. In general, such proteins have a molecular weight below 120,000 Dalton and include amelogenins, non-amelogenins, proline-rich non-amelogenins and tuftelins.

Examples of proteins for use according to the invention are amelogenins, proline-rich non-amelogenins, tuftelin, tuft proteins, serum proteins, salivary proteins, ameloblastin, sheathlin, and derivatives thereof, and mixtures thereof. Moreover, other proteins for use according to the invention are found in the marketed product EMDOGAIN® (BIORA AB, Sweden).

EMDOGAIN® (BIORA AB, S-205 12 Malmo, Sweden) contains 30 mg enamel matrix protein (EMD), heated for 3 hours at about 80° C. in order to inactivate residual proteases, and 1 ml Vehicle Solution (Propylene Glycol Alginate), which are mixed prior to application, unless the protein and the vehicle are tested separately. The weight ratio is about 80/8/12 between the main protein peaks at 20, 14 and 5 kDa, respectively.

In general, the major proteins of an enamel matrix are known as amelogenins. They are markedly hydrophobic substances that under physiologically conditions form aggregates. They may carry or be carriers for other proteins or peptides.

A presently preferred embodiment of the present invention therefore relates to a pharmaceutical, cosmetic and/or therapeutic formulation and/or composition comprising at least one of each polypeptide fragment of amelogenin, as disclosed by the present invention.

Another presently preferred embodiment of the present invention relates to a pharmaceutical, cosmetic and/or therapeutic formulation and/or composition consisting of at least one of each polypeptide fragment of amelogenin, as disclosed by the present invention.

The fraction and/or the at least one of each N-terminal polypeptide fragment of amelogenin, as disclosed by the present invention may in the context of the present invention, be in a substantially isolated or purified form. It will be understood that the fractions, proteins, polypeptides, peptides and/or fragments thereof may be mixed with carriers or diluents or be comprised in a pharmaceutical composition, which will not interfere with the intended purpose of the proteins, polypeptides, peptides and/or fragments thereof and which will still be regarded as substantially isolated. Such a substantially purified form will generally comprise the Fraction Consisting of and protein, polypeptide, peptide and/or fragment in a preparation in which more than 90%, e.g. 95%, 96%, 97%, 98% or 99% of the protein in the preparation is a Fraction and/or a combined polypeptide fragment according to the invention.

By a protein, polypeptide, peptide and/or fragment thereof having an amino acid sequence at least, for example 95% identical to a reference amino acid sequence, is intended that the amino acid sequence of e.g. the polypeptide is identical to the reference sequence, except that the amino acid sequence may include up to 5 point mutations per each 100 amino acids of the reference amino acid sequence. In other words, to obtain a polypeptide having an amino acid sequence at least 95% identical to a reference amino acid sequence: up to 5% of the amino acids in the reference sequence may be deleted or substituted with another amino acid, or a number of amino acids up to 5% of the total amino acids in the reference sequence may be inserted into the reference sequence. These mutations of the reference sequence may occur at the amino and/or carboxy terminal positions of the reference amino acid sequence or anywhere between those terminal positions, interspersed either individually among amino acids in the reference sequence or in one or more contiguous groups within the reference sequence.

In the present invention, a local algorithm program is best suited to determine identity. Local algorithm programs, (such as Smith-Waterman) compare a subsequence in one sequence with a subsequence in a second sequence, and find the combination of subsequences and the alignment of those subsequences, which yields the highest overall similarity score. Internal gaps, if allowed, are penalized. Local algorithms work well for comparing two multidomain proteins, which have a single domain or just a binding site in common.

Methods to determine identity and similarity are codified in publicly available programs. Preferred computer program methods to determine identity and similarity between two sequences include, but are not limited to, the GCG program package (Devereux, 3 et al (1994)) BLASTP, BLASTN, and FASTA (Altschul, S. F. et al (1990)). The BLASTX program is publicly available from NCBI and other sources (BLAST Manual, Altschul, S. F. et al, Altschul, S. F. et al (1990)). Each sequence analysis program has a default scoring matrix and default gap penalties. In general, a molecular biologist would be expected to use the default settings established by the software program used.

The proteins of an enamel matrix can typically be divided into a high molecular weight part and a low molecular weight part, which Fraction Contains acetic acid extractable proteins generally referred to as amelogenins (cf. EP-B-0 337 967 and EP-B-0 263 086).

By separating the proteins, e.g. by precipitation, ion-exchange chromatography, preparative electrophoresis, gel permeation chromatography, reversed phase chromatography or affinity chromatography, the different molecular weight amelogenins can be purified.

As mentioned above the fraction C for use according to the invention typically has a molecular weight of between approximately 4 and 6 kDa, such as approximately SkDa, as determined by SDS PAGE electrophoresis.

In general, the enamel matrix, enamel matrix derivatives and enamel matrix proteins are hydrophobic substances, i.e. less soluble in water, especially at increased temperatures. In general, these proteins are soluble at non-physiological pH values and at a low temperature such as about 4-20° C., while they will aggregate and precipitate at body temperature (35-37° C.) and neutral pH.

In a specifically preferred embodiment, a formulation for use according to the present invention, thus comprises active enamel substances which at least partially are aggregated, and/or which after application in vivo are capable of forming aggregates. The particle size of said aggregates being in a range of from about 1 μm to about 20 nm, such as between 1 μm and 20 nm, 1 μm and 10 nm, 5 μm and 10 nm, 10 μm and 1 nm, 100 nm and 10 nm, 100 μm and 1 nm, 1 μm and 1 nm, 1 μm and 5 nm, 1 μm and 15 nm.

In accordance to the present invention the isolated fraction C and/or the isolated fraction of enamel matrix proteins, which consists of at least one of each two polypeptide fragments of amelogenin as shown in SEQ. ID. No:1 and 2, or a pharmaceutical preparation, which comprises at least one of each two polypeptide fragments of amelogenin as shown in SEQ. ID. No:1 and 2, produced naturally by alternate splicing or processing, or by either enzymatic or chemical cleavage of a natural length protein, or by synthesis of polypeptides in vitro or in vivo (e.g. recombinant DNA methods and/or cultivation of diploid cells), or to at least one of each two isolated polypeptide fragments of amelogenin as shown in SEQ. ID. No:1 and 2, produced naturally by alternate splicing or processing, or by either enzymatic or chemical cleavage of a natural length protein, or by synthesis of polypeptides in vitro or in vivo (e.g. recombinant DNA methods and/or cultivation of diploid cells), may be used together with other active drug substances such as, e.g. anti-bacterial, anti-inflammatory, antiviral, antifungal substances or in combination with local chemotherapy, inducers of apoptosis, growth factors such as, e.g., TGFβ, PDGF, IGF, FGF, EGF, keratinocyte growth factor or peptide analogues thereof. Enzymes—either inherently present in the enamel matrix or preparation thereof or added—may also be used in combination with an enamel matrix fraction and/or polypeptide fragment according to the present invention, especially proteases.

### Pharmaceutical Compositions

Depending on the use of a fraction(s) and/or polypeptide fragments) according to the present invention, a composition may be a pharmaceutical and/or a therapeutic and/or a cosmetic composition. In the following a pharmaceutical and/or therapeutic composition is also intended to embrace cosmetic compositions as well as compositions belonging to the so-called grey area between pharmaceuticals and cosmetics, namely cosmeceuticals.

A pharmaceutical and/or therapeutic composition comprising the fraction(s) and/or polypeptide fragment(s) according to the present invention, serves as a drug delivery system. In the present context the term “drug delivery system” denotes a pharmaceutical and/or therapeutic composition (a pharmaceutical and/or therapeutic formulation or a dosage form) that upon administration presents the active substance to the body of a human or an animal.

For the administration to an individual (such as an animal or a human), fraction(s) and/or polypeptide fragment(s) according to the present invention and/or a preparation thereof, are preferably formulated into a pharmaceutical composition containing the fraction(s) and/or polypeptide fragment(s) of an active enamel substance and, optionally, one or more pharmaceutically acceptable excipients.

A composition comprising a fraction(s) and/or polypeptide fragment(s) according to the present invention to be administered, may be adapted for administration by any suitable route, e.g. by systemic administration to a patient through a hose, syringe, spray or draining device.

Furthermore, a composition may be adapted to administration in connection with surgery, e.g. as a systemic administration by infusion into the blood, lymph, ascites, or spinal fluids, or by inhalation. For systemic application, the compositions according to the invention may contain conventionally non-toxic pharmaceutically acceptable carriers and excipients according to the invention, including microspheres and liposomes. Administration of a composition according to the present invention may also be performed via any other conventional administration route, such as, but not limited to, an oral, parenteral, intravenous, buccal, aural, rectal, vaginal, intraperitoneal, topical (dermal), or nasal route, or by the administration to a body cavity such as e.g. a tooth root or a tooth root canal.

Other applications may of course also be relevant such as, e.g., application on dentures, protheses, implants, and application to body cavities such as the oral, nasal and vaginal cavity. The mucosa may be selected from oral, buccal, nasal, aural, rectal and vaginal mucosa. Furthermore, the application may be directly on or onto a wound or other soft tissue injuries.

Furthermore, application within the dental/odontologic area is also of great importance. Relevant examples are application to periodontal (dental) pockets, to gingiva or to gingival wounds or other wounds located in the oral cavity, or in connection with oral surgery.

A composition for use in accordance with the present invention may be, but is not limited to, in the form of, e.g., a fluid, semi-solid or solid composition such as, but not limited to, dissolved transfusion liquids, such as sterile saline, Ringer's solution, glucose solutions, phosphate buffer saline, blood, plasma, water, powders, microcapsules, bioabsorbable patches, drenches, sheets, bandages, plasters, implants, pills, sprays, soaps, suppositories, vagitories, toothpaste, lotions, mouthwash, shampoo, microspheres, nanoparticles, sprays, aerosols, inhalation devices, solutions, dispersions, wetting agents, suspensions, emulsions, pastes, ointments, hydrophilic ointments, creams, gels, hydrogels (e.g. poly ethylene glycols), dressings, devices, templates, smart gels, grafts, solutions, emulsions, suspensions, powders, films, foams, pads, sponges (e.g. collagen sponges), transdermal delivery systems, granules, granulates, capsules, agarose or chitosan beads, tablets, microcapsules, freeze-dried powders, granules, granulates or pellets, and mixtures thereof.

Suitable dispersing or wetting agents for use in accordance with the invention, may be naturally occurring phosphatides, e.g., lecithin, or soybean lecithin; condensation products of ethylene oxide with e.g. a fatty acid, a long chain aliphatic alcohol, or a partial ester derivable from fatty acids and a hexitol or a hexitol anhydride, e.g. polyoxyethylene stearate, polyoxyethylene sorbitol monooleate, polyoxyethylene sorbitan monooleate, etc. The invention is however not limited thereto.

Suitable suspending agents are, e.g., naturally occurring gums such as, e.g., gum acacia, xanthan gum, or gum tragacanth; celluloses such as, e.g., sodium carboxymethylcellulose, microcrystalline cellulose (e.g. Avicel® RC 591, methylcellulose); alginates and kitosans such as, but not limited to, sodium alginate, etc.

A liquid composition, for use in accordance with the present invention, may e.g. be, but is not limited to, a solution, dispersion or suspension for application on a surface of e.g. a medical implant or device. Once applied, the composition should preferably solidify, e.g. by drying, to a solid or at least highly viscous composition which does not dissolve on storage or when the implant or device is in use.

Such a composition is preferably applied under sterile conditions and/or sterilised after application by irradiation or exposure to ethylene oxide gas. When the composition is in the form of a liquid composition, it may also be applied shortly before the medical implant or device is to be introduced into the body. As an alternative to applying a composition comprising a fraction(s) and/or polypeptide fragments) of an active enamel substance on the medical implant or device, the composition may be applied on a surface of a tissue which is in contact with the implant or device, such as a tissue comprising a substantial proportion of epithelial cells as indicated above. Furthermore, the composition may be applied on both the implant and/or device and on a tissue in contact therewith.

It should also be emphasized that any other pharmaceutical composition as disclosed by the present invention may be used for the application on a surface of a medical implant or device.

A composition according to the present invention, may also, in addition to what already has been disclosed herein, be formulated according to conventional pharmaceutical practice, see, e.g., “Remington's Pharmaceutical Sciences” and “Encyclopedia of Pharmaceutical Technology”, edited by Swarbrick, 3. & 3. C. Boylan, Marcel Dekker, Inc., New York, 1988.

A pharmaceutically acceptable excipient is a substance which is substantially harmless to the individual to which the composition is to be administered. An excipient is comprised in a pharmaceutical composition according to the invention. Such an excipient normally fulfils the requirements given by the national health authorities. Official pharmacopoeias such as e.g. the British Pharmacopoeia, the United States of America Pharmacopoeia and The European Pharmacopoeia set standards for pharmaceutically acceptable excipients.

The choice of pharmaceutically acceptable excipient(s) in a composition, and the optimum concentration thereof, for use according to the invention, cannot generally be predicted and must be determined on the basis of an experimental evaluation of the final composition.

However, suitable excipients for the present purpose may be selected from such excipients that promote application of the composition comprising fraction(s) and/or polypeptide fragment(s) according to the present invention on a surface of the implant or device, or that promote the adherence of the composition to the surface on application, or that prevent immediate dissolution of the composition or protract the release of fraction(s) and/or polypeptide fragment(s) according to the present invention from the composition. A person skilled in the art of pharmaceutical formulation can find guidance in e.g., “Remington's Pharmaceutical Sciences”, 18th Edition, Mack Publishing Company, Easton, 1990.

Whether a pharmaceutically acceptable excipient is suitable for use in a pharmaceutical composition is generally dependent on which kind of dosage form is chosen for use for a particular kind of wound, and/or any other type of disorder and/or damage to a body.

The pharmaceutically acceptable excipients may include solvents, buffering agents, preservatives, humectants, chelating agents, antioxidants, stabilizers, emulsifying agents, suspending agents, gel-forming agents, ointment bases, penetration enhancers, perfumes, powders and skin protective agents. It should however be emphasized that the invention is not limited thereto.

Examples of such solvents for use in a composition in accordance with the present invention, are water, alcohols, vegetable or marine oils (e.g. edible oils like almond oil, castor oil, cacao butter, coconut oil, corn oil, cottonseed oil, linseed oil, olive oil, palm oil, peanut oil, poppy seed oil, rape seed oil, sesame oil, soybean oil, sunflower oil, and tea seed oil), mineral oils, fatty oils, liquid paraffin, polyethylene glycols, propylene glycols, glycerol, liquid polyalkylsiloxanes, or other hydrophilic or etheric solvents such as weak acids with a pH of about 5.5-6.0 facilitating the subsequent application of filling materials in the tooth, as well as mixtures thereof.

Examples of buffering agents are citric acid, acetic acid, tartaric acid, lactic acid, hydrogen phosphoric acid, bicarbonates, phosphates, diethylamine etc.

Suitable examples of preservatives are parabens, such as methyl, ethyl, propyl p-hydroxybenzoate, butylparaben, isobutylparaben, isopropylparaben, potassium sorbate, sorbic acid, benzoic acid, methyl benzoate, phenoxyethanol, bronopol, bronidox, MDM hydantoin, iodopropynyl butylcarbamate, EDTA, benzalconium chloride, and benzylalcohol, or mixtures of preservatives.

Examples of humectants are glycerin, propylene glycol, sorbitol, lactic acid, urea, and mixtures thereof.

Examples of chelating agents are sodium EDTA and citric acid.

Examples of antioxidants are butylated hydroxy anisole (BHA), ascorbic acid and derivatives thereof, tocopherol and derivatives thereof, cysteine, and mixtures thereof.

Examples of emulsifying agents are naturally occurring gums, e.g. gum acacia or gum tragacanth; naturally occurring phosphatides, e.g. soybean lecithin, sorbitan monooleate derivatives; wool fats; wool alcohols; sorbitan esters; monoglycerides; fatty alcohols; fatty acid esters (e.g. triglycerides of fatty acids); and mixtures thereof.

Examples of suspending agents are e.g. celluloses and cellulose derivatives such as, e.g., carboxymethyl cellulose, hydroxyethylcellulose, hydroxypropylcellulose, hydroxypropylmethylcellulose, microcrystalline cellulose, carraghenan, acacia gum, arabic gum, tragacanth, and mixtures thereof.

Examples of gel bases, viscosity-increasing agents or components which are able to take up exudate from a wound are: liquid paraffin, polyethylene, fatty oils, colloidal silica or aluminium, zinc soaps, glycerol, propylene glycol, tragacanth, carboxyvinyl polymers, magnesium-aluminium silicates, Carbopol®, hydrophilic polymers such as, e.g. starch or cellulose derivatives such as, e.g., carboxymethylcellulose, hydroxyethylcellulose and other cellulose derivatives, water-swellable hydrocolloids, carragenans, hyaluronates (e.g. hyaluronate gel optionally containing sodium chloride), collagen, gelatine, pectin, chitosans and alginates including propylene glycol aginate.

In the present invention, fraction(s) and/or polypeptide fragment(s) according to the present invention can be incorporated into a polymeric matrix so that it is released by degradation of the polymeric matrix, by enzymatic action and/or by diffusion. Said polymeric matrix is either suitable for cellular in-growth, or cell-occlusive. Comprised in the invention is thus in particular a pharmaceutical and/or cosmetic formulation of a fraction(s) and/or polypeptide fragment(s) according to the present invention at a low total concentration within the formulation, wherein a spatial and/or selective regulation of release of said active enamel substance permits a great percentage of the active enamel substance to be released at the time of appropriate cellular activity.

Consequently, one aspect of the present invention relates to a pharmaceutical and/or therapeutic formulation for administering a fraction and/or polypeptide fragment according to the present invention, comprising a polymeric matrix, either suitable for cellular growth, in-growth and/or migration, or being cell-occlusive, and a fraction and/or polypeptide fragment, wherein said matrix is formed by a nucleophilic addition reaction between a strong nucleophile and a conjugated unsaturated bond, or a conjugated unsaturated group.

Preferably, the conjugated unsaturated groups or conjugated unsaturated bonds are acrylates, vinylsulfones, methacrylates, acrylamides, methacrylamides, acrylonitriles, vinylsulfones, 2- or 4-vinylpyridinium, maleimides, or quinones.

Examples of ointment bases are e.g. beeswax, paraffin, cetanol, cetyl palmitate, vegetable oils, sorbitan esters of fatty acids (Span), polyethylene glycols, and condensation products between sorbitan esters of fatty acids and ethylene oxide, e.g. polyoxyethylene sorbitan monooleate (Tween).

Examples of hydrophobic or water-emulsifying ointment bases are paraffins, vegetable oils, animal fats, synthetic glycerides, waxes, lanolin, and liquid polyalkylsiloxanes.

Examples of hydrophilic ointment bases are solid macrogols (polyethylene glycols).

Other examples of ointment bases are triethanolamine soaps, sulphated fatty alcohol and polysorbates.

Examples of powder components are: alginate, collagen, lactose, powder which is able to form a gel when applied to a wound (absorbs liquid/wound exudate). Normally, powders intended for application on large open wounds must be sterile and the particles present must be micronized.

Examples of other excipients are polymers such as carmelose, sodium carmelose, hydroxypropylmethylcellulose, hydroxyethylcellulose, hydroxypropylcellulose, methylcellulose, pectin, xanthan gum, locust bean gum, acacia gum, gelatin, carbomer, emulsifiers like vitamin E, glyceryl stearates, cetanyl glucoside, collagen, carrageenan, hyaluronates and alginates and kitosans.

Examples of diluents and disintegrating agents are but not limited to lactose, saccharose, emdex, calcium phosphate materials, such as calcium phosphate substrates, calcium phosphate carriers (comprising hydroxyapatite, bi-phasic calcium phosphates, and tri-calcium phosphates), calcium carbonate, calcium sulphate, mannitol, starches and microcrystalline cellulose.

Examples of binding agents are, but not limited to, saccharose, sorbitol, gum acacia, sodium alginate, gelatine, starches, cellulose, sodium coboxymethylcellulose, methylcellulose, hydroxypropylcellulose, polyvinylpyrrolidone and polyetyleneglycol.

Compositions which have proved to be of importance in connection with topical application are those which have tixothropic properties, i.e. the viscosity of the composition is affected e.g. by shaking or stirring so that the viscosity of the composition at the time of administration can be reduced and when the composition has been applied, the viscosity increases so that the composition remains at the application site.

However, it is appreciated that in those cases where a pharmaceutically acceptable excipient may be employed in different dosage forms or compositions, the application of a particular pharmaceutically acceptable excipient is not limited to a particular dosage form or of a particular function of the excipient.

In a toothpaste or mouthwash formulation or other formulation for application to teeth or tooth roots, fraction(s) and/or polypeptide fragment(s) according to the present invention may either be present in a dissolved state in a vehicle of slightly acid pH or as a dispersion in a vehicle of neutral pH. It is anticipated that fraction(s) and/or polypeptide fragment(s) according to the present invention may form a protective layer on the surface of the teeth, thereby preventing the attachment of caries producing bacteria. In such dental care preparations, the fraction and/or polypeptide fragment may be formulated together with one or more other compounds which have a caries preventive effect, notably fluorine or another trace element such as vanadium or molybdenum. At neutral pH, the trace element is believed to be bound to (e.g. by ion bonds) or embedded in the active enamel substance from which it is released to exert its caries preventive effect when the fraction and/or polypeptide fragment is dissolved at a pH of about 5.5 or less, e.g. due to acid production by caries producing bacteria.

In a pharmaceutical composition for use according to the invention, fraction(s) and/or polypeptide fragment(s) according to the present invention is generally present in a concentration ranging from about 0.01% to about 99.9% w/w. The amount of composition applied will normally result in an amount of total protein per cm² area of dental pulp corresponding to from about 0.005 mg/mm² to about 5 mg/mm² such as from about 0.01 mg/mm² to about 3 mg/mm².

In those cases where the fraction(s) and/or polypeptide fragment(s) according to the present invention is administered in the form of a liquid composition, the concentration of the fraction and/or fragment in the composition is in a range corresponding to from about 0.01 to about 50 mg/ml, e.g. from about 0.1 to about 30 mg/ml. Higher concentrations are in some cases desirable and can also be obtained such as a concentration of at least about 100 mg/ml.

Defect areas in dental pulp in humans typically have a size of about 5-10×2−4×5-10 mm corresponding to about 200 μl and normally at the most about 0.5-1 ml such as about 0.2-0.3 ml per tooth is applied of a composition having a concentration of about 1-40 mg total protein/ml such as, e.g., 5-30 mg/ml is applied. 0.2-0.3 mg/ml corresponds to about 6 mg protein per 25-100 mm² or about 0.1 mg/mm² if calculated only on root surface. Normally an excessive volume is applied to cover the affected surfaces adequately. Even a multilayer would only require a small fraction of the above-mentioned amounts.

### BRIEF DESCRIPTION OF THE FIGURES

FIG. 1. RT-PCR of cultured osteoblasts (one donor, NHO-3). A1 stimulates expression of the following gene products to a greater extend than A2, most prominently after 7 days: osteocalcin and leptin. Values represent the relative concentrations of each protein relative to α-tubulin and are shown as the means of the single results from duplicate experiments.

FIG. 2. RT-PCR of two osteosarcoma cell cultures (SaOS-2 and HOS) stimulated with fractions A1 and A2: Expression of ALP and OC increased, especially by treatment with A1. No effect was observed on the expression of Cbfa-1 and CD44 with A1, whereas expression of CD44 was slightly reduced after treatment with A2. n=3, values represent the relative concentrations of each protein relative to α-tubulin and are shown as the mixed means from the two cell lines results with ±SD.

FIG. 3a. SDS PAGE and Western blot EMD vs. fraction C (Tricine system). Western blot of antibody raised against antigen code EPO42543 in rabbit SK3184 (Peptide=SYGYEPMGWLH corresponding to C terminal of TRAP). 3b. Native charge density page of EMD and fraction C in the presence of Urea.

FIG. 4. Osteocalcin levels. Top Panel: EMD and rhAmel caused a biphasic increase in osteocalcin production. Bottom Panel: Similarly Fraction A and Fraction C had a biphasic effect. For EMD, rhAmel and Fraction A, maximal increases were at 1 ug/ml and for Fraction C, maximal increases were at 10 ug/m|. Data are from one of two experiments and are means±SEM for N=6 independent cultures per variable. Both experiments showed similar results. *p<0.05, treatment v. control.

FIG. 5: DNA content. Effect of different concentration of EMD, recombinant Amelogenin (rhAmel) and Fraction C on DNA levels in MG63 cells. Each experiment was done 2 times and each sample is the Mean and SEM of 6 samples.

FIG. 6: Effect of different concentration of EMD, recombinant Amelogenin (rhAmel) and Fraction C on Alkaline phosphatase (ALP) specific activity in MG63 cells. Each experiment was done 2 times and each sample is the Mean and SEM of 6 samples. Significance * Vrs. no treatment (control) Vrs. 1 μg/□μ□□ p<0.05.

FIG. 7: Effect of different concentration of EMD, recombinant Amelogenin (rhAmel) and Fraction C on osteocalcin level in MG63 cells. Each experiment was done 2 times and each sample is the Mean and SEM of 6 samples. Significance * Vrs. no treatment (control) Vrs. 1 μg/□μ□□ p<0.05.

FIG. 8: Effect of different concentration of EMD, recombinant Amelogenin (rhAmel) and Fraction C on OPG level in MG63 cells. Each experiment was done 2 times and each sample is the Mean and SEM of 6 samples. Significance * Vrs. no treatment (control). Vrs. 1 μg/□μ□□ p<0.05.

FIG. 9: Effect of different concentration of EMD, recombinant Amelogenin (rhAmel) and Fraction C on VEGF level in MG63 cells. Each experiment was done 2 times and each sample is the Mean and SEM of 6 samples. Significance * Vrs. no treatment (control). Vrs. 1 μg/□μ□□ p<0.05.

FIG. 10: Ratio of OC to TP. in % of control

FIG. 11A+B: Mass spectrometry of active components C1RP

FIG. 11C: Mass spectrometry of active components C2RP

FIG. 12: EMD fractionation. Fractions tested in hPDL cells, Pulp cells, hMSC and NHO cells.

FIG. 13: Amelogenin processing

FIG. 14A+B+C+D: EMD cellular uptake experiments. 148. PDL cells after 16 hours incubation with FITC labelled EMD. 14C. PDL fibroblasts, 6 hours incubation, Fluorescein labelled 20 kD Amelogenin. 14D. PDL fibroblasts specifically take up the 20 kDa full length amelogenin and digest it into TRAP. Cellular uptake is mostly through phagocytosis. The 5 kDa fragment seems to locate in the nucleus. The PDL cells do not take up any 5 kDa molecule from the medium.

FIG. 15A: Proliferation capacity based on the measurement of Bromodeoxyuridine (BrdU) incorporation during DNA synthesis. MG63 cells were labeled with BrdU after 24 hours of treatment. B: Proliferation and viability detected by WST-1 assay. MG63 call viability was determined after 7 days of treatment. C: Alkaline phosphatase activity of treated MG63 monolayer cultures after 1 day. ALP activity in μM Pi/min/mg protein.

FIG. 16: PDL cell attachment after 4 and 8 hours

FIG. 17: Table 3: Real time RT-PCR confirmed gene expression changes of Affymetrix analysis of primary osteoblasts 24 h after stimulation with EMD (50 ug/ml) in comparison to PTH1 84 (10-8M). There is a remarkable similarity between effects of the two different agents. Values from 1 donor cell culture, means from two analyses per condition.

FIG. 18: Table 5: Overview of changes in gene expressions by human osteoblasts and mesenchymal stem cells (Cfu-f) assayed with real time RT-PCR with focus on osteoblast differentiation markers. The values for Cfu-f represent the results from 1 donor cells (Cfu-f) only. Statistical analysis has been performed with SIGMA plot software, Spearman Rank order test.

FIG. 19: Table 6: Overview of changes in protein expressions by human osteoblasts and mesenchymal stem cells (Cfu-f) assayed with ELISA with focus on osteoblast-osteoclast communication factors. The values represent the mean values from at least 2 donors in duplicate analyses.

### DETAILED DESCRIPTION

### Experimental Section

### Methods

Separation of Fractions from EMD.

The separation and purification of the different fractions from complete EMD has been performed in serial HPLC column processes:
1) Size exclusion HPLC (TKSSW 2,000-600×30) in Acetonitrile (ACN)/0.9% NaCl
2) Reverse phase HPLC (YMC-C8, 250×20 mm), in a linear gradient of 30% ACN/0.9% NaCl to 60% CAN/0.9% NaCl,

After this step, with a reverse phase HPLC column (like in step 1)), the components A (including the two distinct components A1 and A2, both of which are >20 kD and recognized by anti-amelogenin antibodies (conventional antibodies against EMD, BIORA AB, SE), B, B1, B2, B3, FRACTION C, C3, C4, D, D2 were showing as distinct peaks and could be separated by fractionating.
2)a To separate 4 distinct subfractions (Cp1, Cp2, Cp3, Cp4) from the FRACTION C fraction, a reverse phase HPLC column was used (ACE-C4 with a linear ACN-acidic gradient) (Advanced Chromatography Technology, USA)
3) The last step was intended to remove ACN from the samples by using a HI-Trap desalting column (HR 16/20) (Ammersham Biosciences, SE). To determine concentrations of the fractions, a size exclusion HPLC column was utilized (same as in step 1)) with PBS).

### Cells Used for the Experiments.

Commercially available primary human osteoblasts from both femur and tibia of different donors (NHOst cell system; Cambrex, Walkersville, Md., USA) were grown in osteoblast growth media (OGM, Cambrex). Cultured osteoblasts were exposed to hydrocortisone hemisuccinate (200 nM) and h-glycerophosphate (10 mM) (Cambrex) in ambient medium to facilitate mineralization. The phenotype of cells was characterized based on the expression levels of alkaline phosphatase (ALP), collagen type 1, osteocalcin, and CD44 (late differentiation marker), and formation of mineralization nodules.

Human periodontal ligament cells (PDL, pooled from 3 donors, Biowhittaker, Passage 6), were treated as described elsewhere (Gestrelius, S., Andersson, C., Lidstrom, D., Hammarstrom, L. & Somerman, M. In vitro studies on periodontal ligament cells and enamel matrix derivative. 3 Clin Periodontol 24, 685-92 (1997)).

The osteosarcoma cell line SaOS-2 (ATTC HTB-85) was obtained from American Type Culture Collection (Rockville, Md.). SaOS-2 cells were grown in McCoy's 5A medium (PM) supplemented with 10% FCS and 1% penicillin-streptomycin solution.

The mouse osteoblastic cell line MC3T3-E1 was obtained from Deutsche Sammlung von Mikroorganismen and Zellkulturen (DSMZ No ACC 210; Braunschweig, Germany) and maintained in a-MEM (PAA, Linz, Austria) containing 20 mM HEPES, 10% FCS (PAA), and 1% penicillin-streptomycin solution.

The monoclonal osteosarcoma cell line, OHS, was a kind gift from Dr. Bruland (The Norwegian Radium Hospital, Oslo, Norway). The cells were cultured in RPMI 1640 (PAA) with 10% FCS (PAA), 50 IU/ml penicillin, and 50 g/ml streptomycin.

Colony-forming-unit fibroblasts (Cfu-f), human mesenchymal stem cells which were separated and characterized by flow analysis cytometry (FACS) were obtained from the bone marrow of two voluntary donors (Radium hospital, Oslo) and treated in the same way as the osteoblasts (mentioned above).

### SDS PAGE and Western Blotting.

16% SDS Tricine gels, loaded 0.5 μg FRACTION C and 5 μg EMD per lane. Western blot of antibody raised against antigen code EPO42543 in rabbit SK3184 (antigen peptide ═SYGYEPMGWLH, corresponding to C-terminal of TRAP), 1st Ab diluted 1:500, 2nd Ab (anti Rabbit biotin conjugate, SIGMA) diluted 1:1,000, avidin alkaline phosphatase conjugate diluted 1:20,000 (SIGMA).

### Conventional RT-PCR.

Traditional endpoint techniques were used as described elsewhere (Reseland, J. E. et al. Leptin is expressed in and secreted from primary cultures of human osteoblasts and promotes bone mineralization. 3 Bone Miner Res 16, 1426-33 (2001)).

### Real Time RT PCR.

Cells were cultured after treatment with 5 ug/ml peptides or FRACTION C (50 ug/ml EMD respectively) for 24 h, 4d and 7d and messenger RNA was extracted using magnetic beads (Dynal A S. Oslo, Norway) and cDNA was synthesized from cell lysates according to the manufacturer's instructions (iScript One-Step RT-PCR with SYBR Green, BIORAD). Real time PCR has been performed according to the manufacturer's protocol (iCycler, BIORAD). Each reaction has been run in duplicate, and results represent the mean values of at least 2 independent cell donors.

Primer pairs were as given in table 2:

TABLE 2

| | | |
|---|---|---|
| CD44 | Fw | 5′-CCCAGATGGAGAAAGCTCTG-3′ |
| | Rev. | 5′-GAAGCAATATGTGTCATACTGGGAG-3 |
| IL-6 | Fw | 5′ CCCACACAGACAGCCACTCACCTC |
| | Rev | 5′ ATACCTCAAACTCCAAAAGACCAG |
| Collagen | Fw | 5′ CAGCCGCTTCACCTACAG |
| | Rev | 5′ TTTTGTATTCAATCACTGTCTTGCC |
| Osteocalcin | Fw | 5′ GAAGCCCAGCGGTGCA |
| | Rev | 5′ CACTACCTCGCTGCCCTCC |
| Cbfa-1 | Fw | 5′ GCCTTCAAGGTGGTAGCCC |
| | Rev | 5′ CGTTACCCGCCATGACAGTA |

### Affymetrix Gene Array.

Primary osteoblasts were cultured in T75 culture flasks and cultured after treatment for 24 h, 72 h or 7d respectively and lysed in 7 ml Trizol solution (Gibco, USA). Trizol lysates were kept at −70° C. until RNA isolation according to the manufacturer's protocols and further processing (Dep. of Medical Biochemistry, Oslo University, Norway). Double stranded cDNA and biotin labeled cRNA probes were made from 5 μg total RNA using the Superscript Choice system (Invitrogen) and the Enzo Bioarray respectively. Procedures were according to recommendations from Affymetrix. This cRNA was hybridized to Hu-133A chips (Affymetrix) containing cDNA oligonucleotides representing more than 22,000 transcripts followed by washing and staining on the GeneChips Fluidics Station 450 (Affymetrix) according to manufacturers instructions. The chips were scanned on the Affymetrix GeneArray® 2500 scanner. The quality of the RNA and probe was controlled by an Affymetrix based test measuring the ratio between 5′ and 3′ mRNAs for α-actin and GAPDH and found to be highly satisfactory. The datasets were processed by the Affymetrix Mas5.0 software, and signal values representing the expression level of each transcript were generated. Each procedure has been done in twice in parallel and the resulting values indicate the mean of at least two donor's cells in duplicate experiments.

### Affymetrix Experiments Performed so Far:

### 1. Leptin—PTH—EMD

### 2. Leptin—PTH—EMD—FRACTION C

3. rat recombinant Amelin—EMD—FRACTION C

### Animal Experiments.

### 1) Minipig Experiments: Fraction C in Peg Gels

### Aim

Study to investigate the efficacy of frcation C on mandibular bone formation.

3 minipigs (2 pigs 12-18 months, 1 adult pig >18 months)

Surgery: Flap of the lower jaw Q3 and Q4, drilled cavities with a diameter of 10 mm Treatments:
5 defects treated with a collagen sponge impregnated with fraction C
3 defects treated with fraction C in PEG-thiol mixed with PEG-acrylate in Cekol buffer
2 defects as a negative control with collagen sponges with PBS
1 defect with sham surgery

Fixation in 4% paraformaldehyde (in situ prep) for 24 hours.

Demineralisation: in 0.5 M EDTA pH 8.0 (in situ prep) for 8-12 weeks. Changing buffer 2 times a week. Histology: After about 12 weeks sectioning and staining for in situ hybridisation and histology.

### 3) Rat Calvaria Model AMO

### Dates and Methods

PEG in combination with fraction C in 12/2005. 2.3 mm diameter defects in the mandibles, 100 ug FRACTION C/ml gel, 4 animals, 2 defects per animal (each side one defect), two time points for healing: 1 and 2 weeks, evaluation by histology
Group 1 week:
1) PEG/fraction C vs. PEG/-
2) PEG/fraction C vs. Collagen/FRACTION C
Group 2 weeks:
3) PEG/fraction C vs. PEG/-
4) PEG/fraction C vs. Collagen/-

### Attachment, Proliferation and Selected Protein Assays.

### Cell Attachment

Cells were seeded in 24 wells plates at a density of 20,000 cells/well in 1 ml medium (MDM medium +10% FCS). For each experimental group n=3. As controls served cells grown without the addition of peptides. The separate peptides were added immediately after cell seeding at a final concentration of 10 μg/ml medium. After 4, 8 and 24 hours respectively the media were aspirated and the wells rinsed with 2×1 ml PBS and the cell layers were trypsinized with 0.2 ml of trypsin/EDTA solution to count attached cells by the use of a CellCounter, Chemometec A/S.

### Cell Proliferation

Cells were seeded in 48 wells plates at a density of 5,000 cells/well/ml medium. For each experimental group n=3. As controls served cells grown without addition of peptides. The separate peptides were added immediately after cell seeding at a final concentration of 10 μg/ml medium. After (4) or 5 days (wells were checked not to be confluent since the proliferation phase otherwise could be passed) cells were detached and counted as for the cell attachment assay.

### Assay for AP-Activity, Protein Content and TGF-β1

Cells were seeded in 48 wells plates at a density of 5000 cells/well/ml medium.

For each experimental group n=3. As controls served medium, cells with medium and peptides in medium without cells (in case of background values). The peptides were added immediately after cell seeding at a final concentration of 10 μg/ml medium. After 5 days (as for the cell proliferation study) the medium from each well was transferred to Eppendorf tubes, centrifugated at 1,000 rpm for 5 min. Supernatants were kept at −20 C until the assays were performed. Cell layers were washed with 2×1 ml PBS. After addition of 0.5 ml of milliQwater to the different cell layers bended pipette tips were used to scrape of the cells. Cell suspensions were transferred to Eppendorf tubes and lysed by the use of an ultrasonic water bath for 10 min and centrifugated at 1000 rpm for 5 min. The cell lysates were kept at −20 C until the assays were performed. Changes of medium (with peptides included as for the initial seeding of cells) were made after 3 days.

### ELISA Immunoassays

For determining proteins in culture supernatants the following kit assays were used:

### Results/Discussion

1. Cell Experiments with Fractions of EMD

### a) A1/A2 (20 kDa Fraction of EMD)

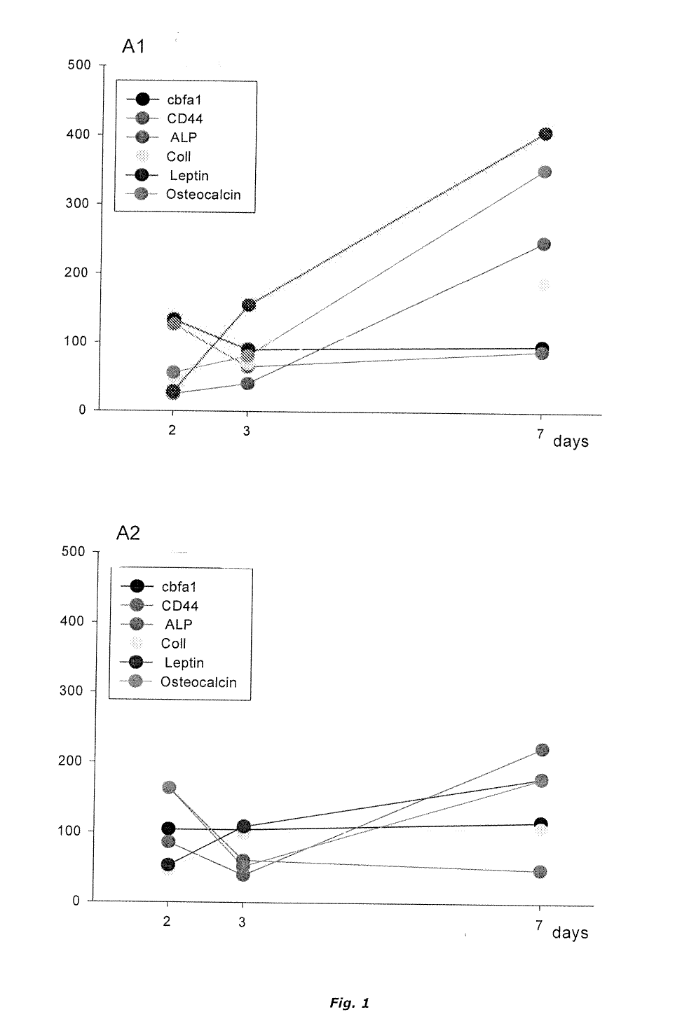

FIG. 1. RT-PCR of cultured osteoblasts (one donor, NHO-3). A1 stimulates expression of the following gene products to a greater extend than A2, most prominently after 7 days: osteocalcin and leptin. Values represent the relative concentrations of each protein relative to α-tubulin and are shown as the means of the single results from duplicate experiments.

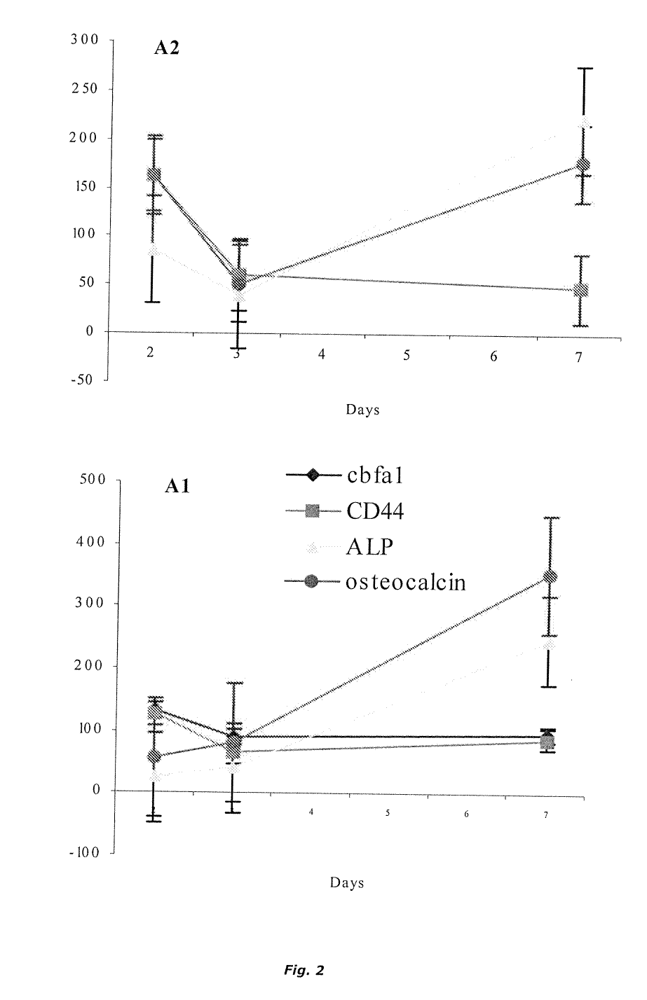

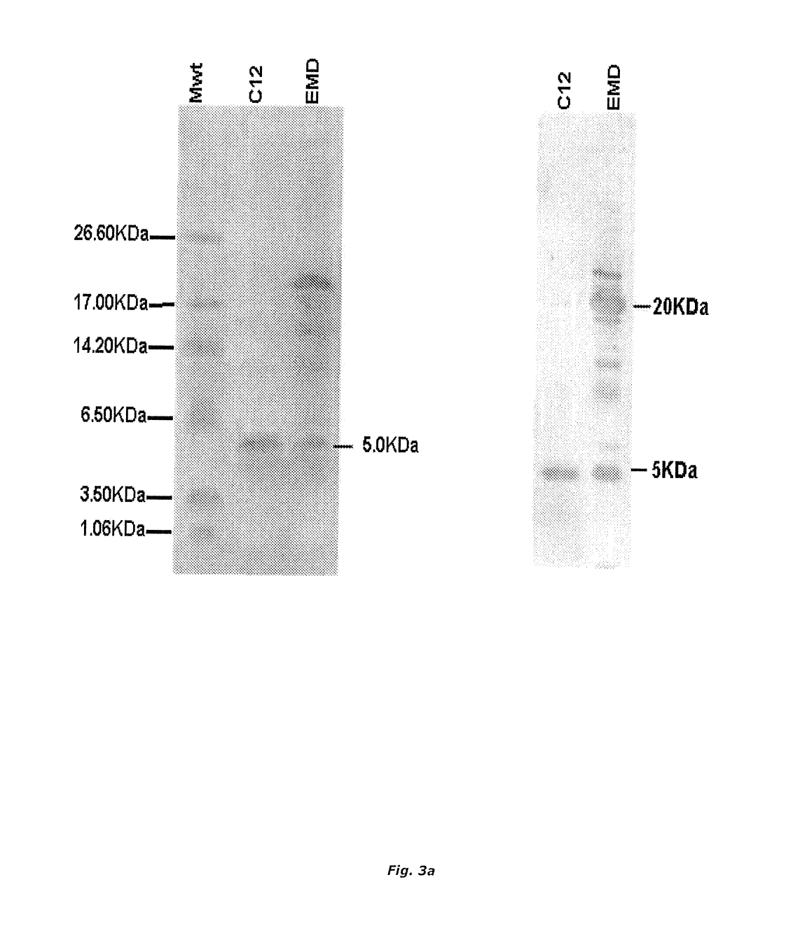

FIG. 2. RT-PCR of two osteosarcoma cell cultures (SaOS-2 and HOS) stimulated with fractions A1 and A2: Expression of ALP and OC increased, especially by treatment with A1. No effect was observed on the expression of Cbfa-1 and CD44 with A1, whereas expression of CD44 was slightly reduced after treatment with A2. n=3, values represent the relative concentrations of each protein relative to α-tubulin and are shown as the mixed means from the two cell lines results with ±SD.

### Summary A1/A2 Gene Expressions

A1 and A2 have different effects on cultured osteosarcoma cells A1 stimulates bone formation by up-regulation of ALP and osteocalcin

A2 reduced the expression of CD44, an osteocyte markerProtein analysis by ELISA With ELISA, an increase of IL-6 secretion, with A1 more potent than A2, has been observed (data not shown). The increase of IL-6 has to be taken carefully into considerations, since IL-6 is a potent pro-inflammatory factor, which can strongly influence effects of other factors.

### b) Fraction C (5 kDa Fraction of EMD)

### I) SDS PAGE and Western Blotting of Fraction C

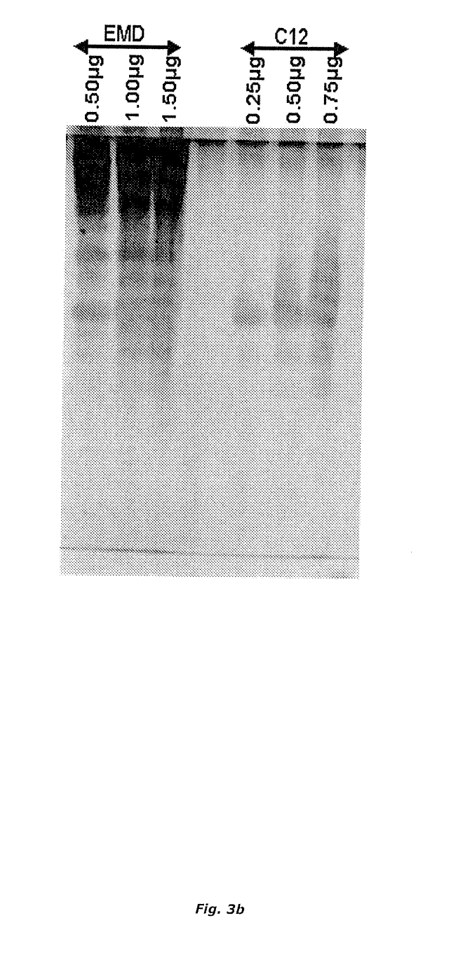

To further characterize the fraction C from EMD, SDS PAGE with subsequent Western blotting was performed. Fraction C showed mainly as a band at around 5 kDa (FIG. 3).

II) Affymetrix Gene Array

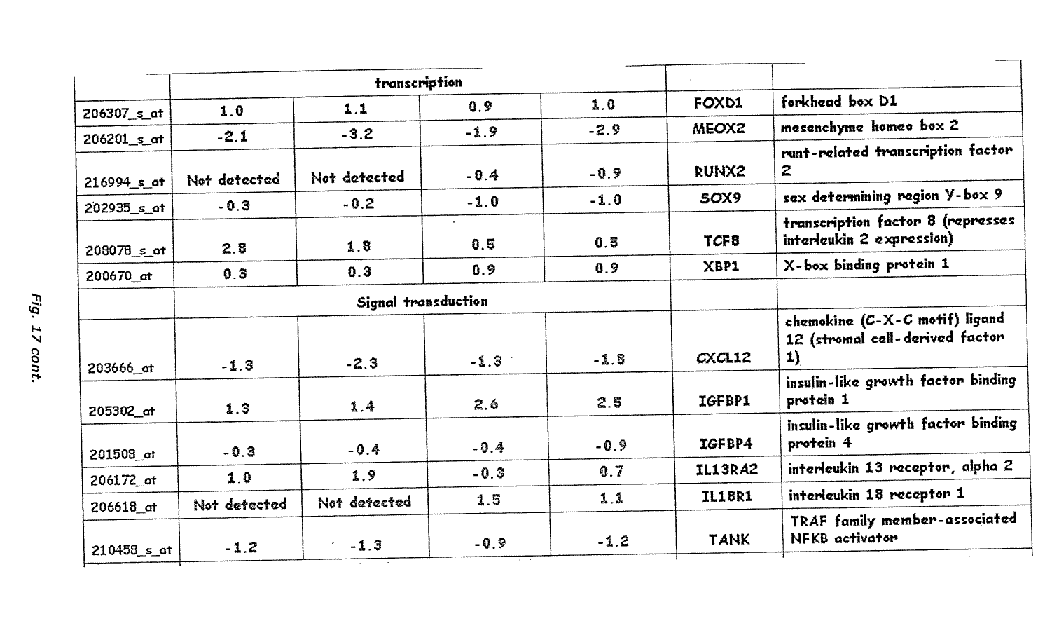

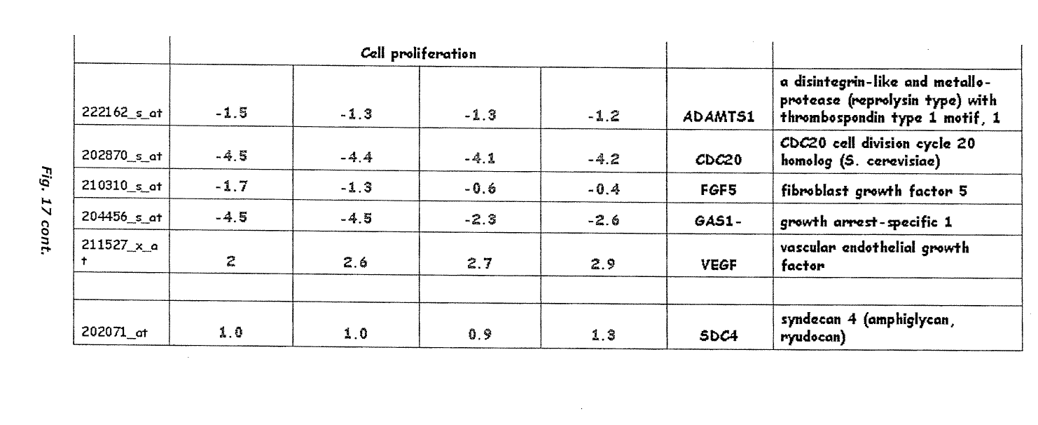

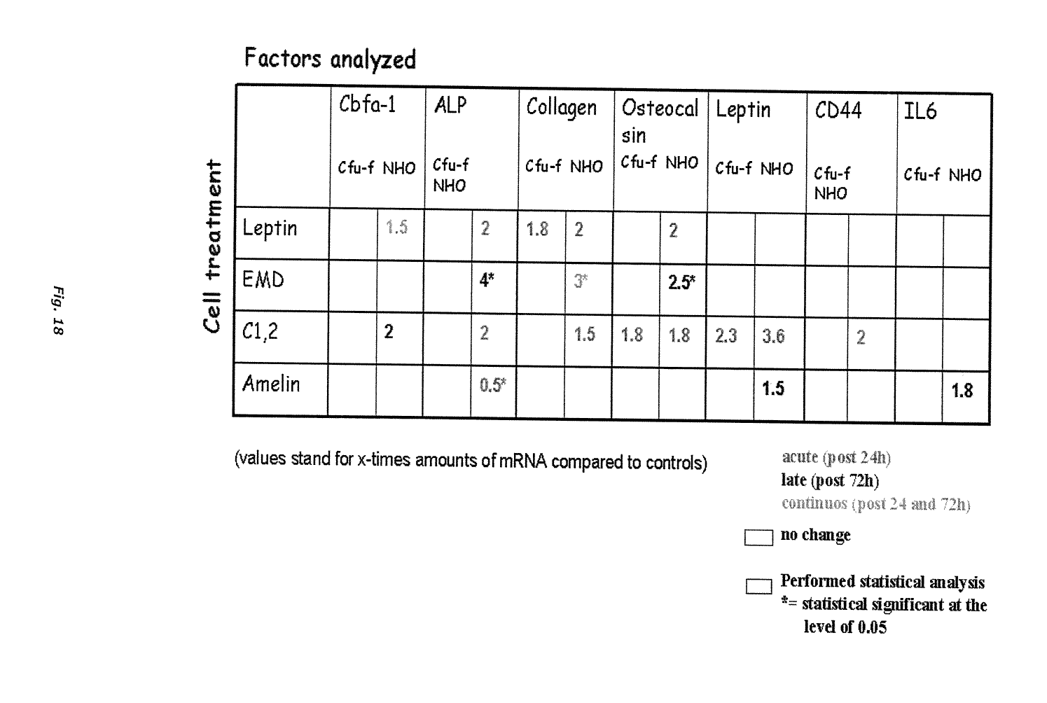

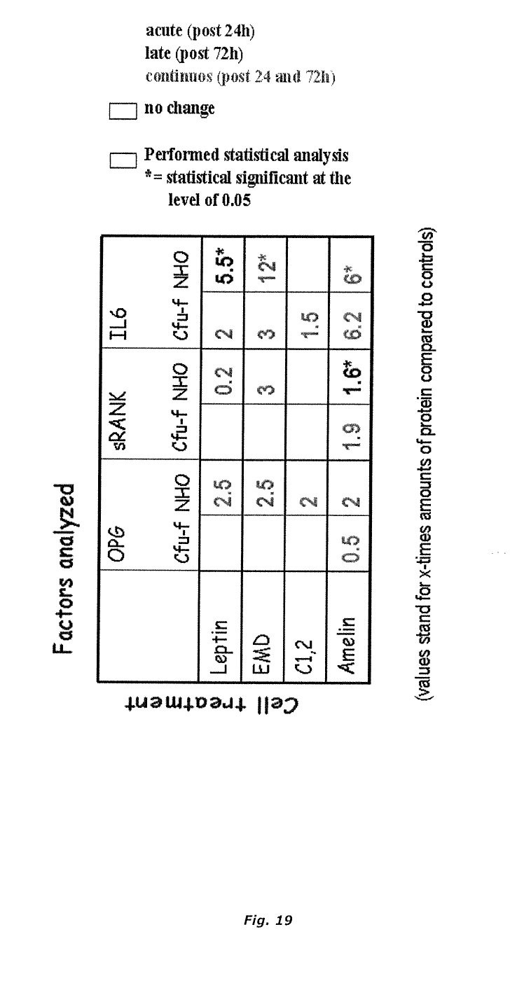

To compare the effects of PTH peptides and EMD on primary osteoblasts, cells of two donors were treated synchronously and expression patterns were analysed by affymetrix gene chip analysis. Interestingly, 24 h after stimulation, expression of 254 genes were more than 2-fold changed in the same direction with both EMD and PTH from non-treated controls. Moreover, no genes were found to be oppositely regulated as much as 2-fold by EMD and PTH. Statistical analysis confirmed a positive correlation between expression changes after stimulation with PTH and EMD (correlation coefficient=0.805; p<0.001). A subset of these gene expression changes were verified by real time RT-PCR confirming the results obtained with Affymetrix analysis (see Table 3/FIG. 17).

### Comparison Effects of Fraction C in Comparison to EMD

To compare effects of fraction C in comparison to EMD, primary osteoblasts from 1 donor (NHO-3) were stimulated with the 5 kDa fraction fraction C and results compared with cells treated with EMD. Log 2 (stimulated/control) values were considered as ≠0 only when ≧|0.5|.

Genes that are Up-Regulated Both by EMD and Fraction C
platelet-derived growth factor receptor, alpha polypeptide (PDGFRA): cell proliferation///platelet-derived growth factor, alpha-receptor activity///ATP binding///transferase activity; integral to plasma membrane
   Genes that are Down-Regulated Both by EMD and Fraction C
fibronectin 1 (FN1): cell motility///cell adhesion///signal transduction; cell adhesion molecule activity extracellular matrix///extracellular space///soluble fraction///Inflammatory_Response_Pathway
   Genes that are Down-Regulated by Fraction C, but not Regulated by EMD
WNT1 inducible signaling pathway protein 1: regulation of cell growth///cell adhesion///signal transduction///cell-cell signaling///cell growth and/or maintenance

integrin, alpha V (vitronectin receptor, alpha polypeptide, antigen CD51): cell-matrix adhesion///integrin-mediated signaling pathway

Genes that are Up-Regulated by EMD but not Regulated by Fraction C
MMP-14 (matrix metalloproteinase 14) (membrane-inserted): proteolysis and peptidolysis
actin, alpha 2, smooth muscle, aorta (ACTA2): muscle development; motor activity///structural constituent of cytoskeleton///structural constituent of muscle; striated muscle thin filament///actin filament
angiopoletin 1: angiogenesis///signal transduction
aggrecan 1 (chondroitin sulfate proteoglycan 1): cell adhesion///heterophilic cell adhesion
vinculin: cell adhesion
dentin sialophosphoprotein (DSPP): extracellular matrix
lamin A/C
thrombospondin 1: cell motility///cell adhesion///development///neurogenesis///blood coagulation
   Genes that are Up-Regulated by EMD but Downregulated by Fraction C
follistatin (FST): development///negative regulation of follicle-stimulating hormone secretion activin inhibitor activity extracellular TGF-beta signaling pathway
wingless-type MMTV integration site family, member 5A (WNT5A): signal transduction///frizzled-2 signaling pathway///cell-cell signaling///development///embryogenesis and morphogenesis///soluble fraction; Wnt_Signaling
   Genes that are Down-Regulated by EMD but not Regulated by Fraction C
CD97 antigen: cell motility///inflammatory response///immune response///cell adhesion///cell surface receptor linked signal transduction///G-protein coupled receptor protein signaling pathway///cell-cell signaling
transforming growth factor, beta receptor II (70/80 kDa) (TGFBR2): protein amino acid phosphory-dation///transmembrane receptor protein serine/threonine kinase signaling pathway///TGF-beta ligand binding to type II receptor///positive regulation of cell proliferation
FGF-2 (fibroblast growth factor 2 (basic)): regulation of cell cycle///activation of MAPK///angiogenesis///chemotaxis///signal transduction///RAS protein signal transduction///cell-cell signaling///histogenesis and organogenesis///neurogenesis///muscle development///cell proliferation
CD14 antigen: phagocytosis///apoptosis///inflammatory response///immune response///cell surface receptor linked signal transduction
leptin receptor: energy reserve metabolism///cell surface receptor linked signal transduction///development
DEAD (Asp-Glu-Ala-Asp) box polypeptide 24

### c) Conventional and Real Time RT-PCR and Protein Expression by Human Osteoblasts

Table 4a) Conventional and real time RT-PCR analysis of human tibia and femur osteoblasts treated with single fractions separated from EMD. The values represent the mean values from two donors in duplicate. Values marked with stars show results from real time RT-PCR, not marked values have been generated by conventional RT-PCR. 4b) Expression of proteins into the culture medium after stimulation with single fractions separated from EMD. The values represent the mean values from two donors in duplicate.

TABLE 4a
gene expression

| | | Colla- | | | | |
|---|---|---|---|---|---|---|
| | ALP | gen | OC | Runx2 | leptin | CD44 |
| EMD | 4 | 3 | 2.5 | | 2 | |
| sol. EMD | 1.5 | 2 | 3.5 | 2 (1.7)* | 6 | 4 (4)* |
| A1 | 2.5 | 2 | 3.7 | 2 | 3 | 3 |
| A2 | 2.3 | | 2 | | 6 | 1.7 |
| B | 1.5 | 2.3 | | | 7 | 1.6* |
| B1 | | | | | | 1.6* |
| B2 | | | 8* | | | 5.6* |
| B3 | | 2 | 3.5 | 2.5 | 7 | 3 (6.7)* |
| C12 | 2 | 1.5 | 2.5 | 2 | 3 (3.6)* | 2 (2.5)* |
| C4 | 1.5 | | | 2 | 3 | 1.8 (1.8)* |
| D | | | 2* | | 2.2* | |
| D1 | | | | | 1.8* | |

(values represent log2 of rel. amounts compared to neg. control)
(values with * are the results from real time RT-PCR)
acute (post 24 h)
late post (72 h)
continuous (post 24 and 72 h)

TABLE 4b
secreted to the culture medium

| | | ALP | Osteo- | | | |
|---|---|---|---|---|---|---|
| | LDH | act. | calcin | sCD44 | IL-6 | OPG |
| EMD | 1.3 | 1.5 | 3 | 4 | 12 | 2.5 |
| sol. EMD | 1 | | 8 | 3 | 30 | 2 |
| A1 | 1 | | 4.3 | 2.5 | 8 | |
| A2 | 1.4 | 2 | 2.5 | 3 | 5.5 | |
| B | | | | | | |
| B1 | | | 1.5 | | | |
| B2 | | | 2.3 | | | |
| B3 | 0.8 | 1.8 | 4 | 10 | 37 | 1.7 |
| C12 | 0.8 | 1.5 | 8 | 2.5 | | 2 |
| C4 | | | | | 8 | |
| D | | | | | | |
| D1 | | | 1.7 | | | 1.6 |

(values represent log2 of rel. amounts compared to neg. control)
acute (post 24 h)
late post (72 h)
continuous (post 24 and 72 h)

### d) ELISA Analysis of Factors Secreted by Human Osteoblasts (NHO) or Mesenchymal Stem Cells (Cfu-f)

Moreover, preliminary experiments with Cfu-f cells stimulated with fraction C and EMD showed for both agents a tendency of reducing expression levels of the pro-inflammatory cytokines IL-1β, TNF-α, and IL-8 (assayed with ELISA, data not shown).

### General Remarks

Some of different fractions from EMD exert effects on different cells in vitro. The focus on fraction C and B3 is based on these experiments showing the most relevant modifications in the expression of genes and proteins related to osteogenesis. First animal experimental results are with fraction C in combination with collagen as a carrier matrix. The use of collagen is controversial, due to its potential to induce inflammation when applied in vivo, but on the other hand, it is the currently best-characterized carrier for this kind of applications.

### Conclusions

First results (n=1) from Affymetrix gene array indicate that EMD seems to be generally more regulation-active for osteoblasts than fraction C. This was confirmed with ELISA analysis of primary osteoblasts for soluble RANK and IL-6, which were both upregulated after exposure to EMD, whereas fraction C (table 5) did not induce such a change in these cells.
However, real time RT-PCR showed a bone-specific upregulation of cbfa-1 in primary human osteoblasts and of osteocalcin in mesenchymal stem cells, whereas EMD did not show this effect (table 4). This indicates a less broad, but rather more osteogenic effect of fraction C compared to EMD. Additionally, the pro-inflammatory potential of fraction C seems to be differentiated compared to that of EMD.

### 2. An In Vitro Study to Evaluate the Effect of Fractions of EMD and Synthetic Peptides on Periodontal Ligament (PDL) Cells

To study the effect of synthetic peptides based on fractions from EMD (SEQ ID NO:1, 2, 3, or 4) on cell proliferation, the activity of AP, and the expression of osteocalcin (OC) and TGF-beta.

### Material

PDL (Periodontal Ligament) cells, pooled from three donors.
Synthesized peptides, 1 mg/ml stock in H2O. Before addition to the cells the peptide solution will be sterile filtrated, 0.22 μm and concentration measured by UV absorption A280 nm. Different concentrations will be prepared by diluting stock solution in sterile 0.1% acetic acid.
EMD lyophilised, dissolved in sterile 0.1% acetic acid.

### Methods

Peptide solutions will be added to the cells at concentrations of 1/5/20/100 μg/ml. EMD (positive control will be applied to the cells at a final conc. of 100 μg/ml (positive control). Cells will be treated with the corresponding volume (to the application of EMD) of 0.1% acetic acid as negative controls.

Cells will be seeded in 24 wells plates at a density of 5,000 cells/well/ml medium in the presence of the stimulating factors for 1, 2 or 5 days in parallel plates. For the group stimulated 5d, the medium will be changed (including stimulating factors) after 2d.

AP activity (early response) will be determined in cell lysates according to standard protocols. Culture supernatants will be collected and frozen at −20° C. until determining concentrations of TGF-beta (early response) and osteocalcin (late response).

Proliferation rates will be estimated by MIT cell proliferation assay.

n=3 for each experimental group.

### 3. 5 kDa Component of Enamel Matrix Derivative Possesses Osteogenic Properties

Analysis of EMD by high performance liquid chromatography revealed the presence of three main components: (1) a 20 kDa protein [Fraction A], (2) two proteins of 12 and 9 kDa, and (3) a 5 kDa peptide [Fraction C]. Two of these components (Fraction A and C) have been purified and characterized. The Fraction A protein corresponds to the full length amelogenin protein and the Fraction C is the N-terminal part of this protein (3). The aim of the present study was to examine the effect of these two EMD components on osteoblasts.

Methods: Confluent cultures of MG63 human osteoblast-like cells and normal human osteoblasts were treated with or without EMD, recombinant human amelogenin (rhAmel), Fraction A (0.01-100 μg/ml) or Fraction C (0.1-250 μg/ml) for 24 hours. Effects on DNA content and alkaline phosphatase specific activity (ALP), and osteocalcin (OCN), osteoprotegerin (OPG), vascular endothelial growth factor A (VEGF-A) and fibroblast growth factor-2 (FGF-2) levels in the conditioned media were determined.

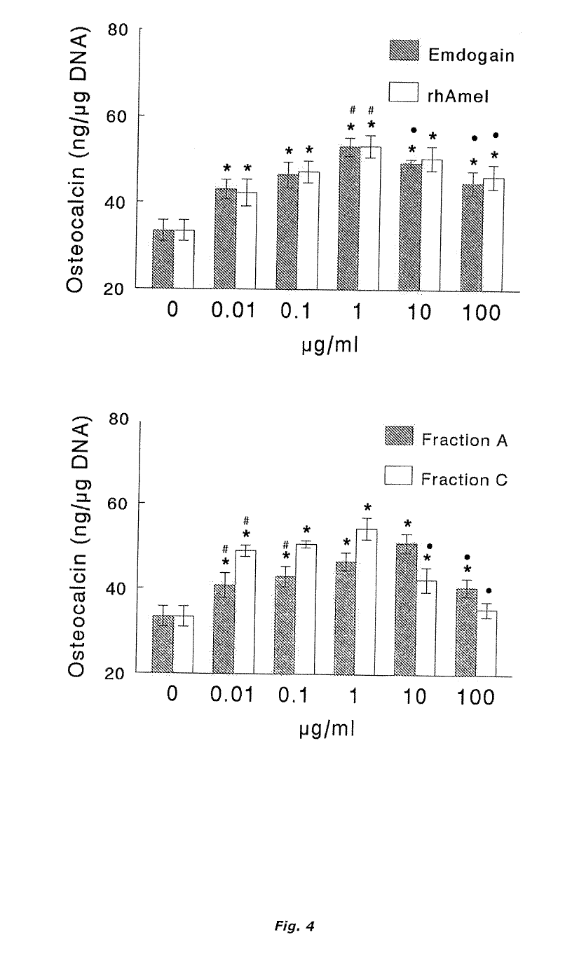

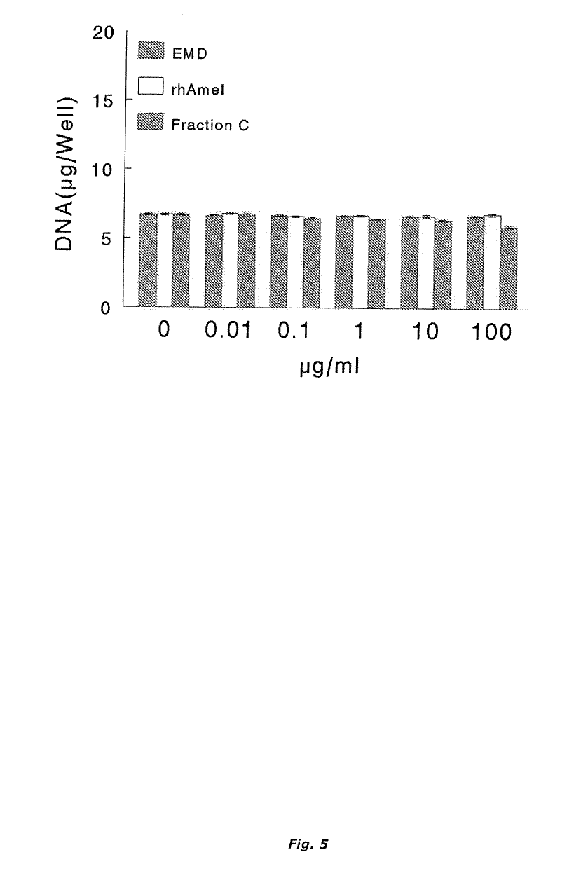

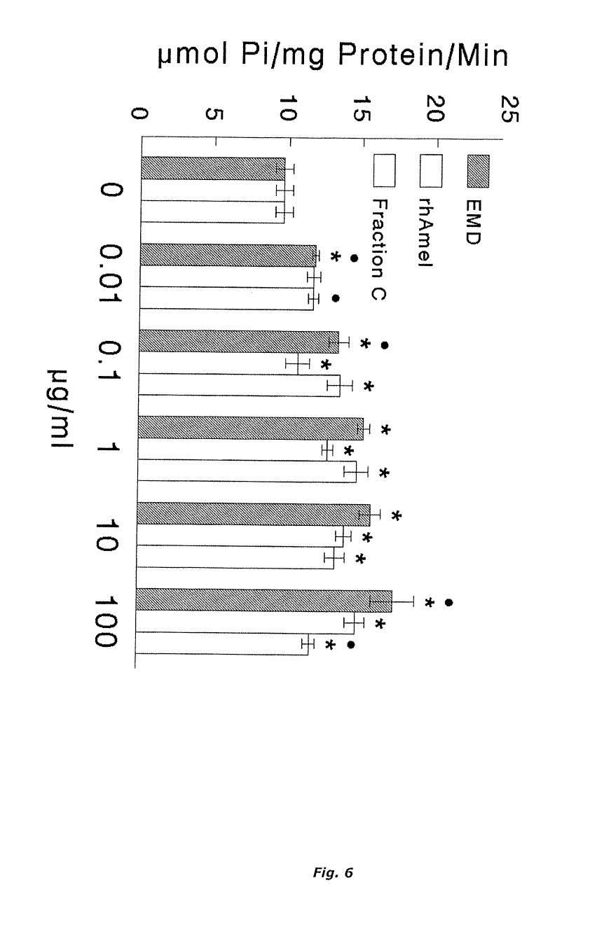

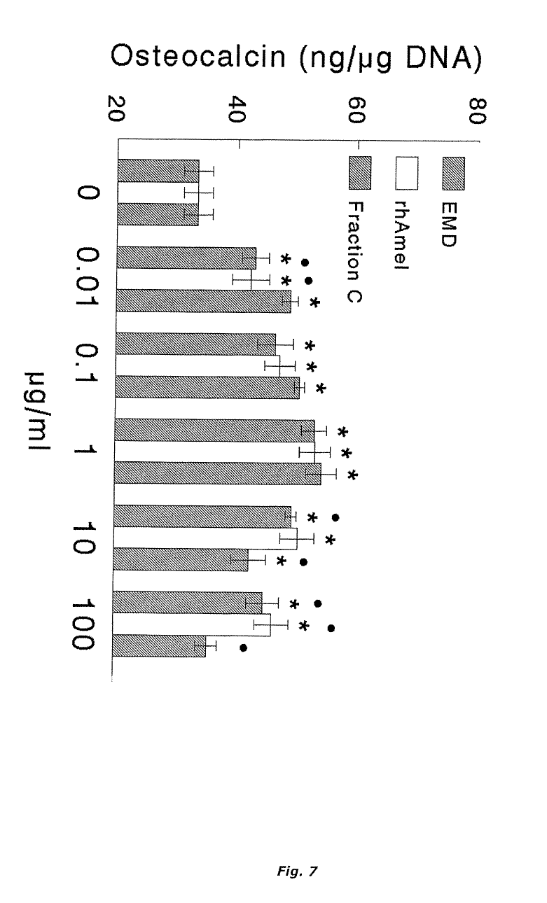

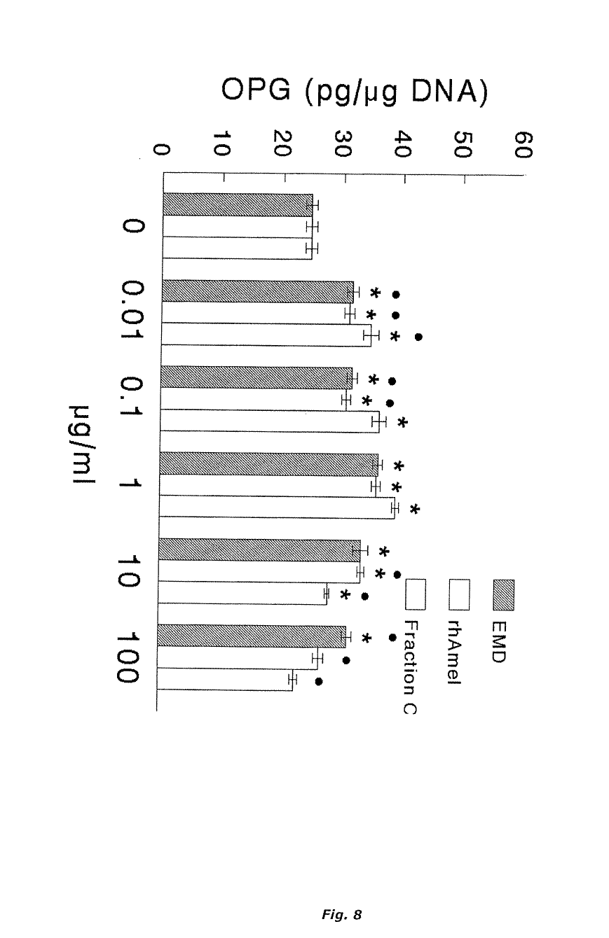

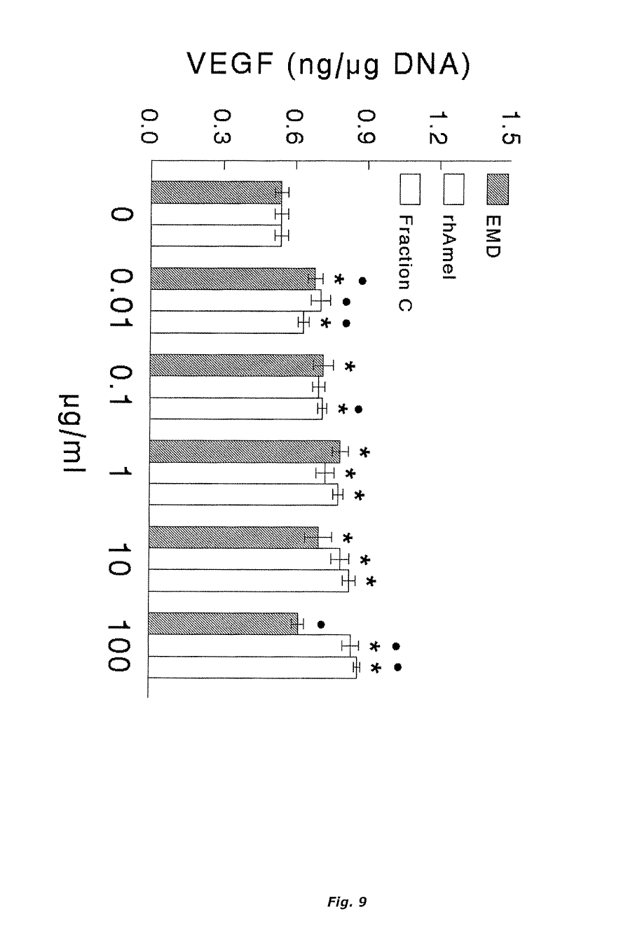

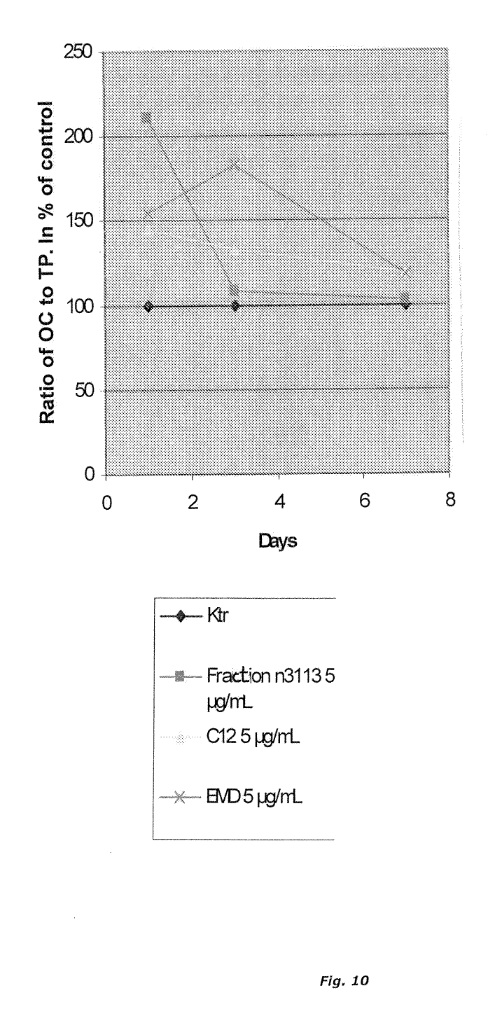

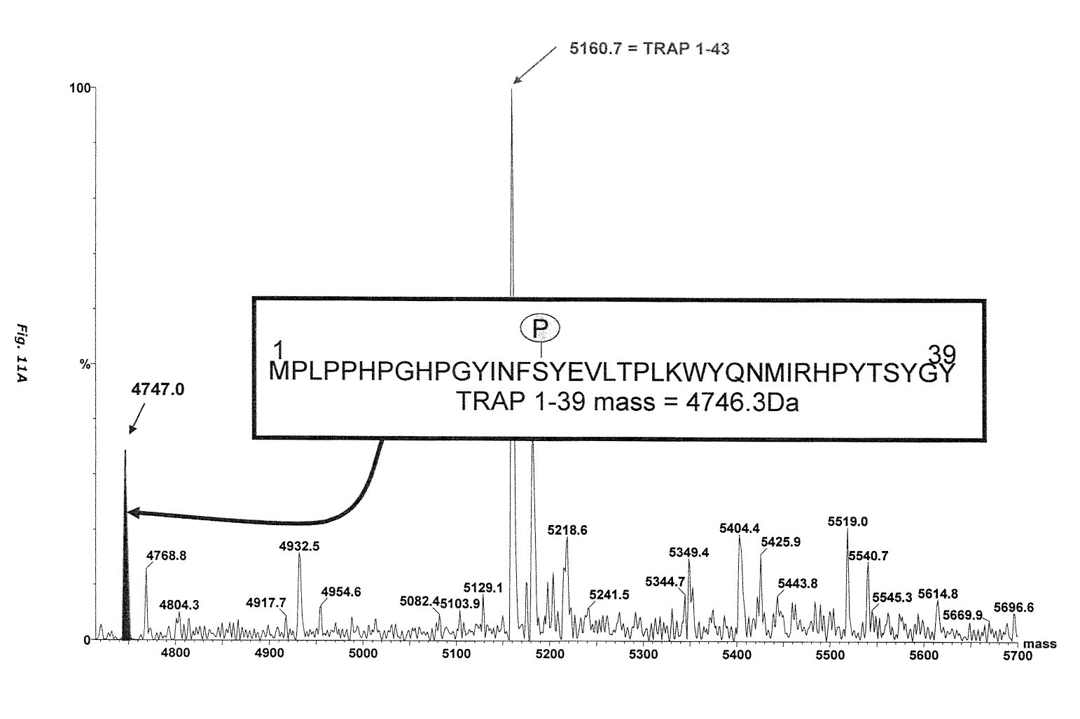

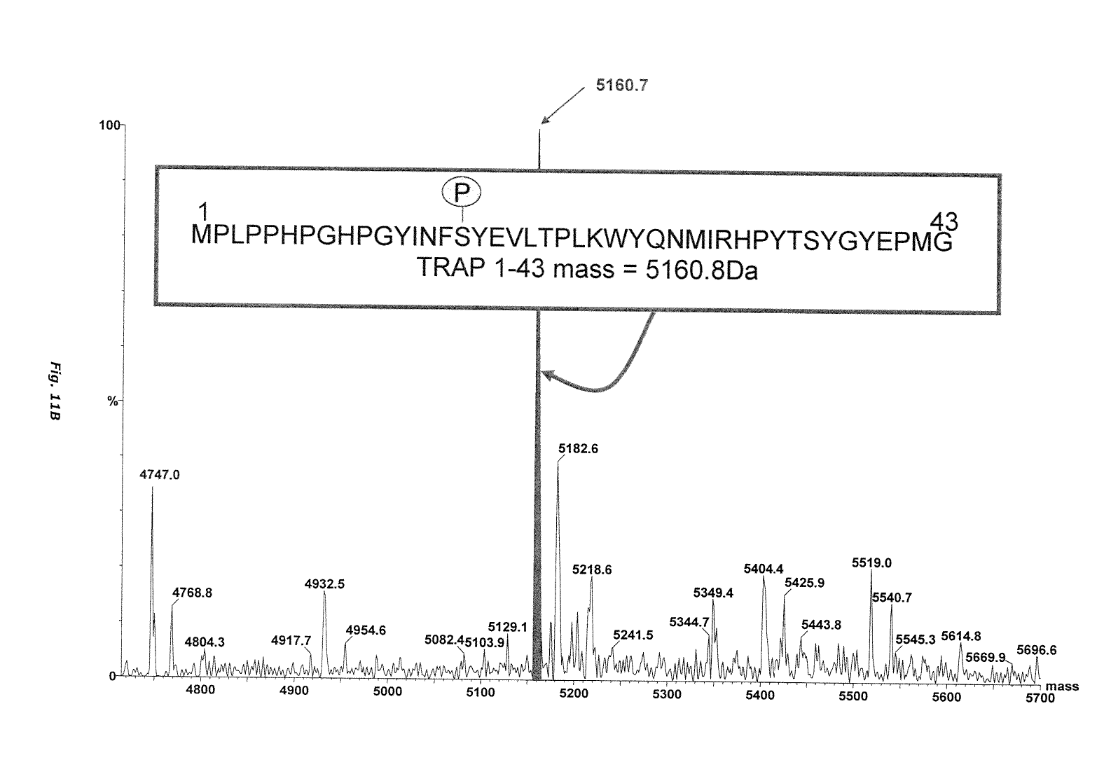

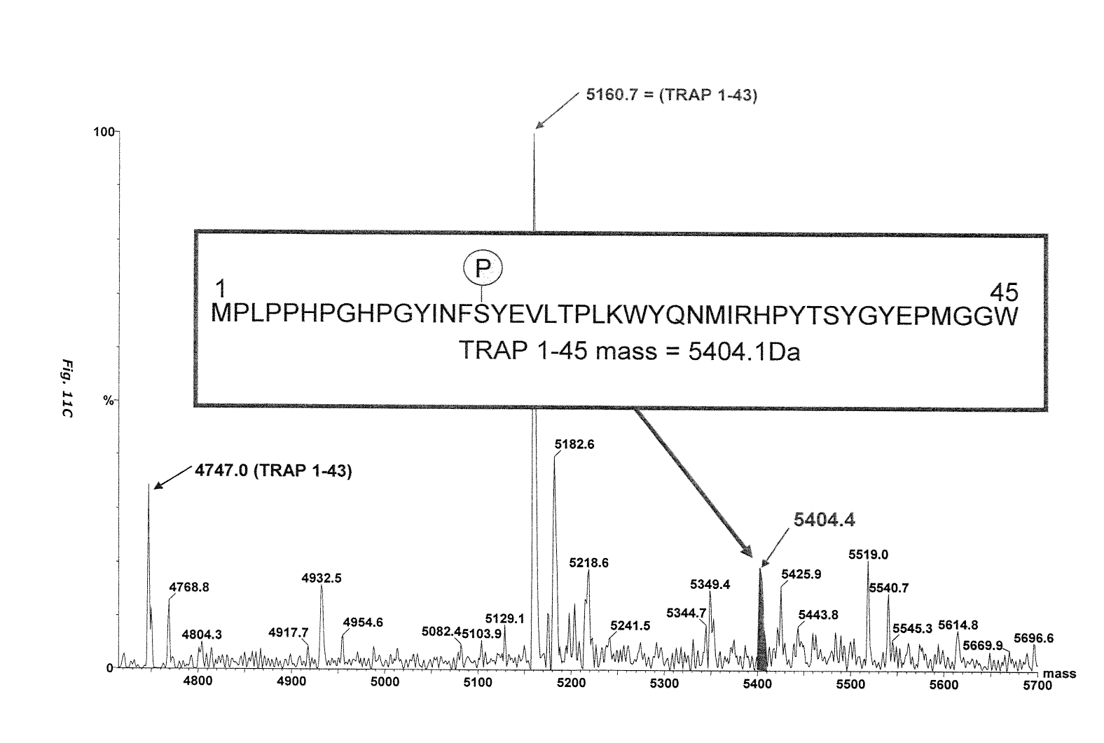

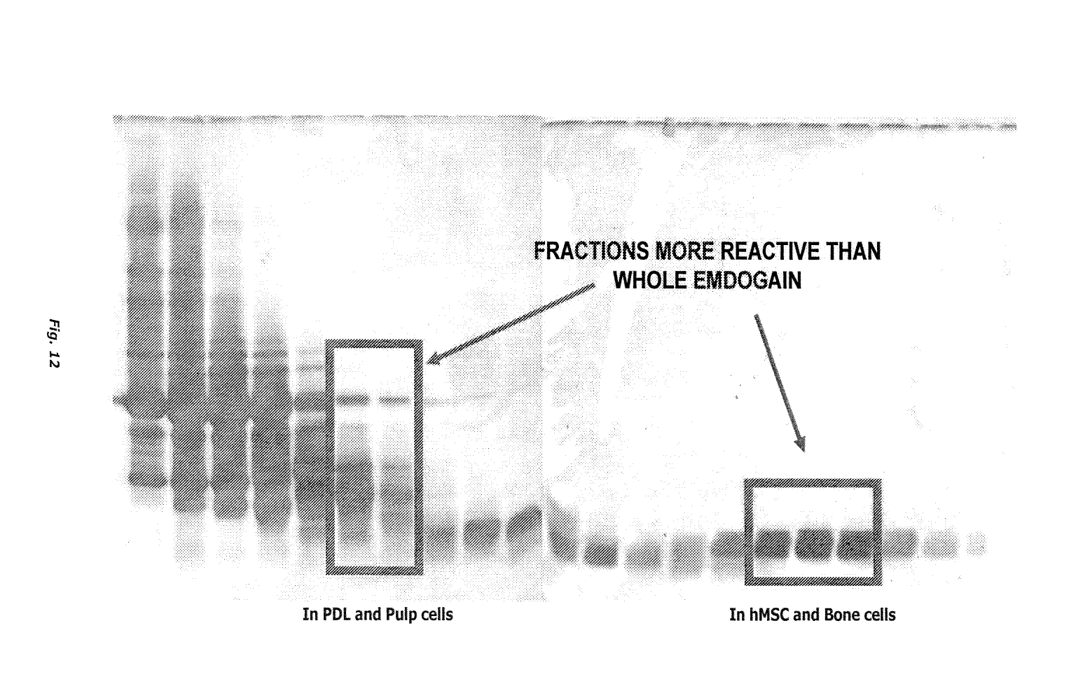

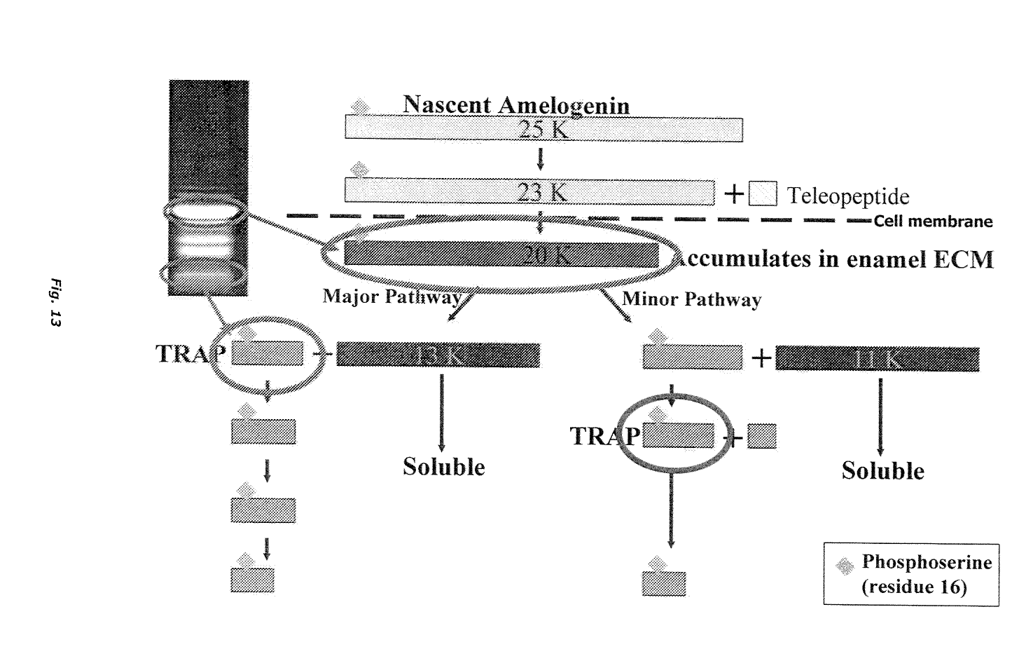

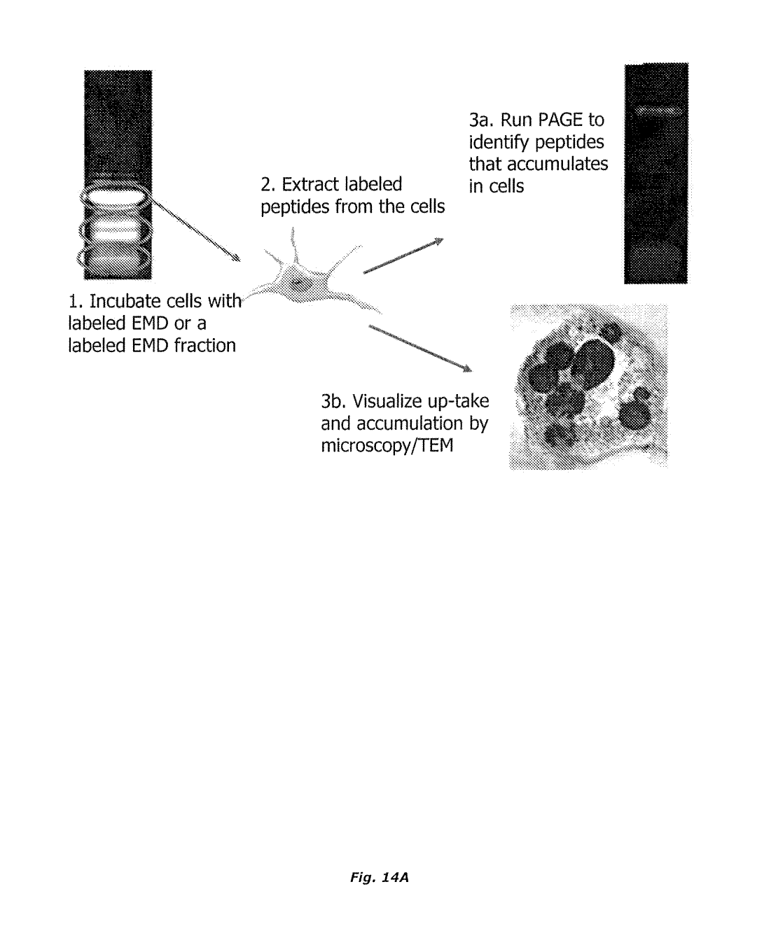

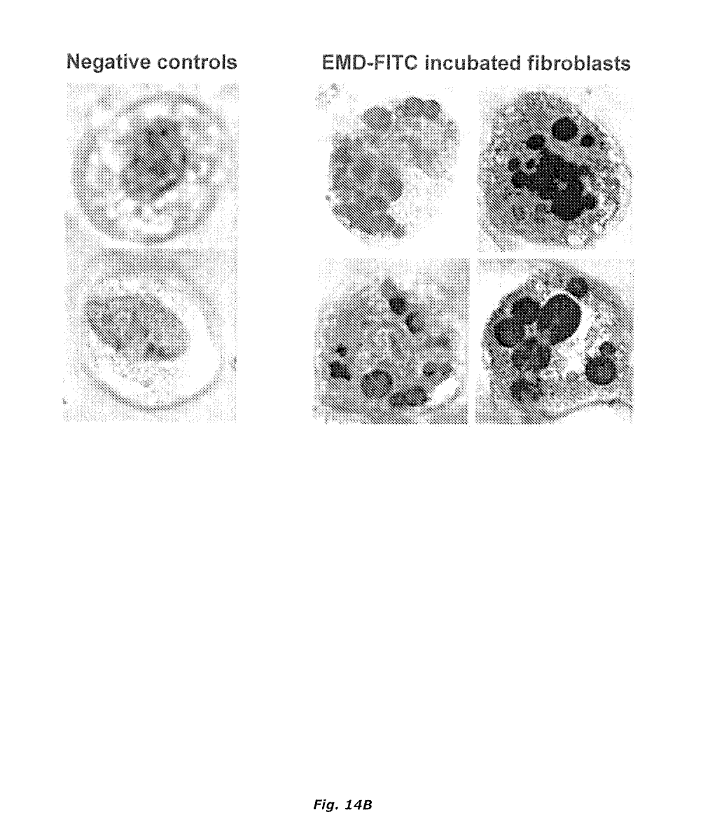

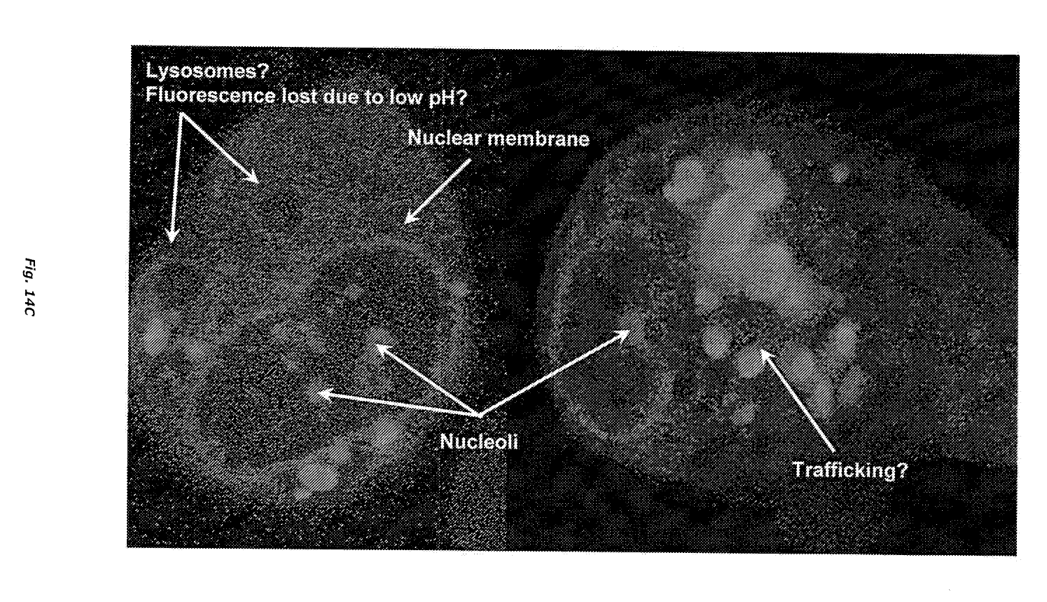

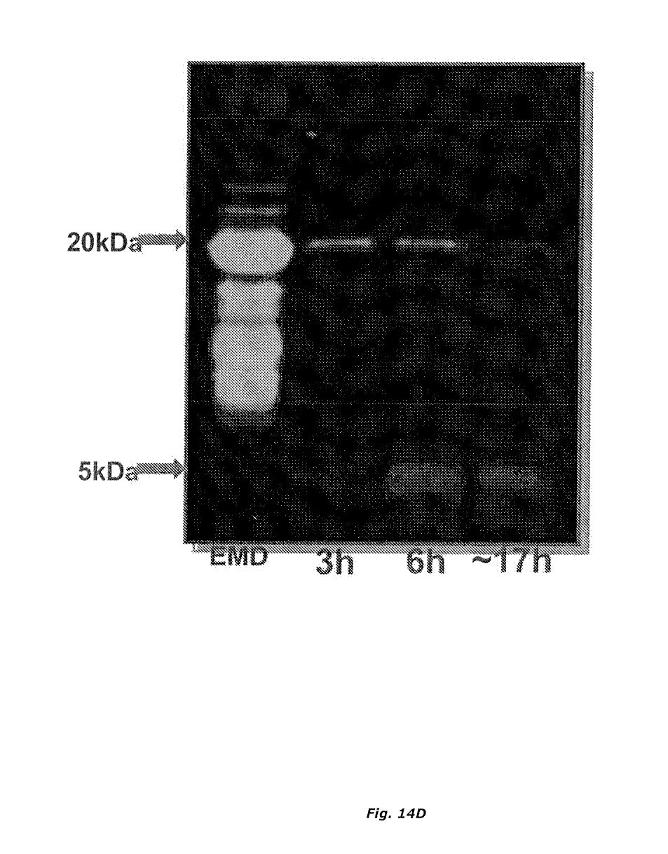

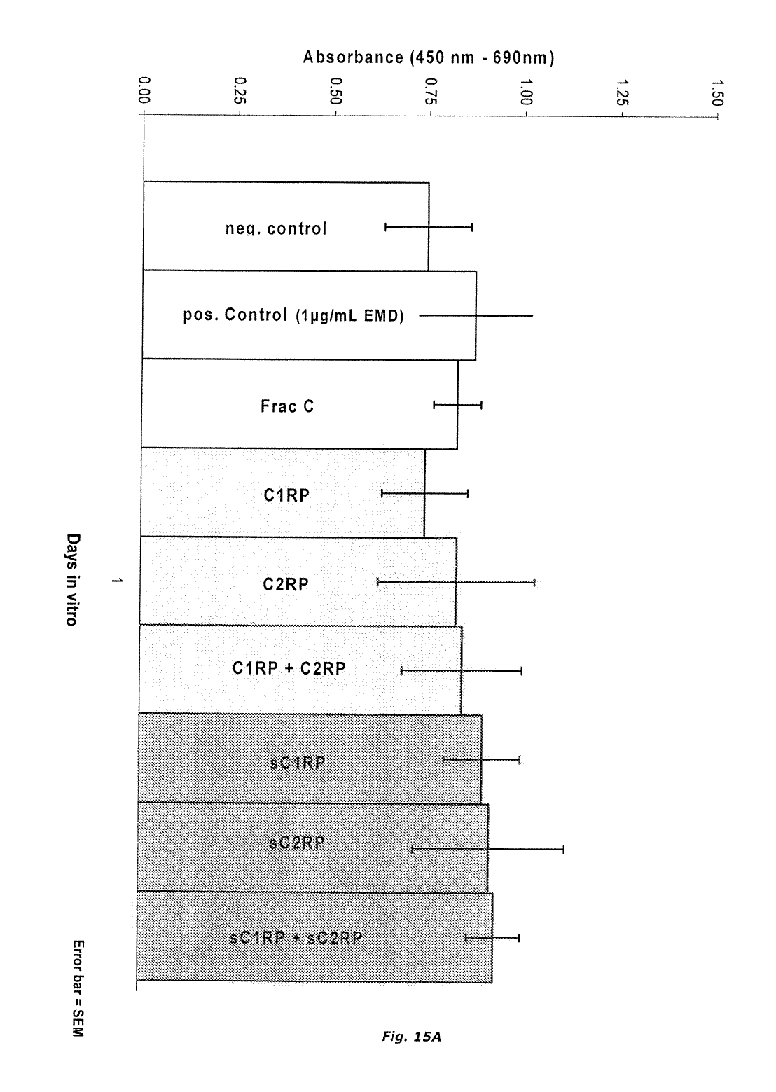

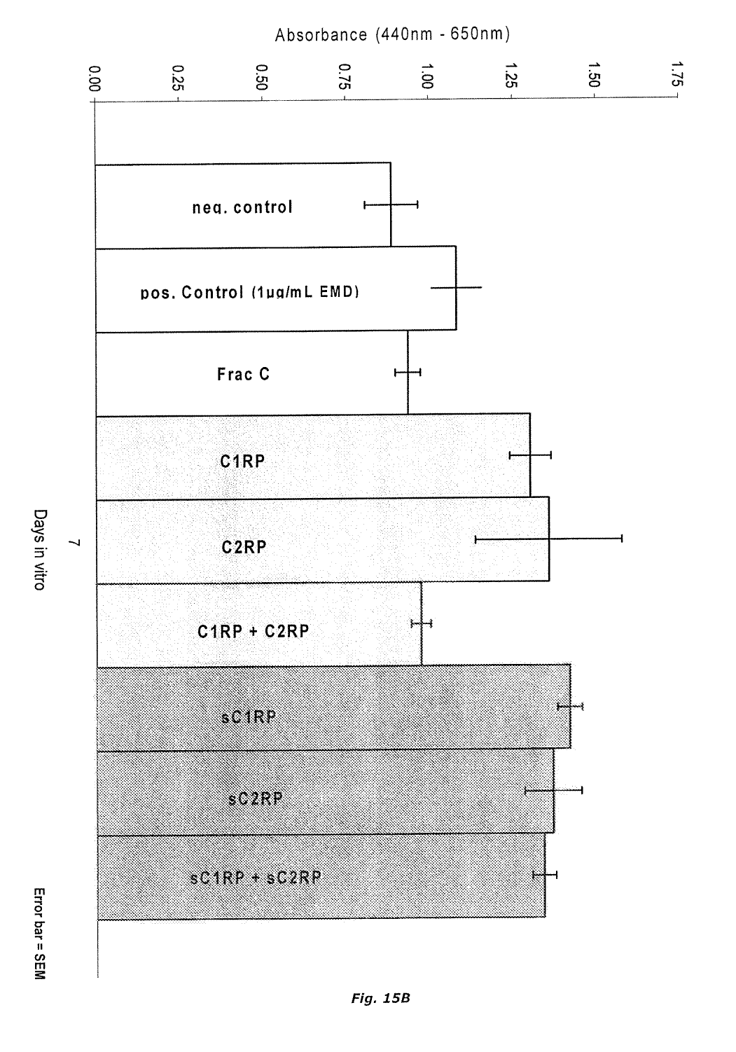

Results: Fraction C reduced DNA content of MG63 cells in a dose-dependent manner and increased osteoblast differentiation markers like alkaline phosphatase and osteocalcin with peak increases at 10 mg/ml. The peptide also increased local factors like OPG, VEGF and FGF in a dose-dependent manner. The effects of the Fraction C were similar to those of the Fraction A, EMD, and rhAmel. Moreover, normal human osteoblasts responded in a similar manner to MG63 cells. See FIG. 4.

Conclusions: These results indicate that the Fraction C component of Emdogain possesses osteogenic activities and that the osteogenic effects of amelogenin may be due the N-terminal region of the protein.

### 4. 5 kDa Component of Enamel Matrix Derivative Induces Osteoblast Differentiation

EMD (Enamel Matrix Derivative) is a protein complex enriched in amelogenins which correspond to the bioactive part of Straumann Emdogain®. Recent published analysis of EMD by High Performance Liquid Chromatography revealed the presence of its three main components: (1) 20 kDa, (2) [12+9] kDa, (3) 5 kDa. Two of these components (20 kDa, 5 kDa) have been purified: the first one (20 kDa) is suspected to correspond to the full length amelogenin protein and the second one (5 kDa) to the N-terminal part of this protein. These two components have been tested in cell cultures comparatively to a recombinant human amelogenin (rhAmel) over-expressed in Escherichia Coli and to the EMD complex.

### Materials and Methods

MG63 osteoblast-like cells, originally isolated from a human osteosarcoma, were obtained from the American Type Culture Collection (Rockville, Md.). These cells are well characterized and exhibit numerous osteoblastic traits, including increased alkaline phosphatase activity and osteocalcin synthesis in response to 1α,25(OH)2D3. Moreover, observations using MG63 cells have been confirmed using normal human osteoblasts, normal mouse calvarial osteoblasts, fetal rat calvarial cells and other osteoblast cell lines, and the results correlate with clinical performance in animals and humans.

MG63 Cells were cultured in Dulbecco's modified Eagle medium (DMEM) containing 10% fetal bovine serum (FBS) and 1% penicillin and streptomycin at 37° C. in an atmosphere of 5% CO2 and 100% humidity. Cells were seeded at 15,000 cells/well, media were changed every 48 h. At confluence the different concentration of the material was added to the culture for 24 We used two determinants of osteoblast differentiation in day-7 cultures: alkaline phosphatase specific activity [orthophosphoric monoester phosphohydrolase, alkaline; E.C. 3.1.3.1] of cell lysates, and osteocalcin content of the conditioned media. cells. Alkaline phosphatase is an early marker of differentiation and reaches its highest levels as mineralization is initiated. Osteocalcin is a late marker of differentiation and increases as mineral is deposited. Lysates were prepared using isolated cells collected by centrifugation after counting. Enzyme activity was assayed by measuring the release of para-nitrophenol from para-nitrophenylphosphate at pH 10.2 and results were normalized to protein content of the cell lysates. The levels of osteocalcin in the conditioned media were measured using a commercially available radioimmunoassay kit (Human Osteocalcin RIA Kit, Biomedical Technologies, Stoughton, Mass.) and normalized to DNA content. The conditioned media from the day-7 cultures were also assayed for growth factors and cytokines. Osteoprotegerin was measured using enzyme-linked immunosorbent assay (ELISA) kit (DY805 Osteoprotegerin DuoSet, R&D Systems, Minneapolis, Minn.). VEGF was assesseds using an ELISA kit (RnD Systems). Briefly, 100 uL of conditioned media was added to precoated plates and incubated for two hours. A specific detection antibody was added to the plate and incubated for an additional two hours. The absorbance of the samples was read using a microplate reader and the results analyzed using a standard curve.

DNA Measurment-Cells were sonicated in 0.5% Trition-X 100 and DNA was measured using Quant-iT™ Pico Green® kit (Invitrogen), which measures double-stranded DNA. The amount of DNA was measured using a fluorescence microplate reader using a DNA standard from 0.2 to 200 ng of DNA.

5. Identification of the Active Compound of Fraction C (5 kda; C1RP or C2RP)

Identification of the active compound of fraction C (5 kDa; C1RP or C2RP).

We have tested synthetic peptides of amelogenin (exon1, 3 and 5) of various lengths.

WBRA001:
MPLPP HPGHPGYINFSYEVLTPLKWYQSIRPP-OH
(MW: 3733.4 g/Mol)
WBRA005:
HPGHPGYINFSYEVLTPLKWYQSIRPP-OH
(MW: 3197.7 g/mol)
WBRA004:
GYINFSYEVLTPLKWYQSIRPP-OH
(MW: 2672.1 g/Mol)
WBRA003:
SYEVLTPLKWYQSIRPP-OH
(MW: 2077.4 g/Mol)
WBRA002:
TPLKWYQSIRPP-OH
(MW: 1485.8 g/Mol)

Fraction C, having a molecular weight of 5250.4 g/mol was used at a final concentration of 5 μg/ml (0.9 μmol/L). The peptides were used in a molar concentration similar to fraction C. WBRA001 enhanced osteocalcin and CD44 expression and secretion similar to fraction C, the smaller peptides had no effect on the bone markers in osteoblasts. 5 kDa amelogenins, isolated from EMD batch 3113 was identified to contain both; C1RP or C2RP (1-43 and 1-45 TRAP), whereas the fraction C isolated from EMD batch 9121 contains not only C1RP and C2R (1-43 and 1-45 TRAP), but 2 further, unidentified minor peaks. The effect of the different isoforms on expression of bone markers was tested on osteoblasts.

The effect on leptin, IL-6 and OPG secretion was similar for the mix of; C1RP and C2RP (3113) and fraction C. The mix (3113) induced an acute (<24 h) release of osteocalcin to the culture medium (figure below). mRNA expression was however increased by fraction C after >3 days of incubation.

The isolated fractions of C1RP and C2RP induced a higher LDH activity in the culture medium than fraction C and the mix of peptides (3113). This slightly more toxic effect on the cells made it difficult to see any difference in effect between these two purified fractions. Conclusion; the mix of C1RP and C2RP (3113) was more potent than fraction C alone in stimulating early osteoblast differentiation. At later stages the Fraction C is a more potent inducer of osteocalcin expression.

### 7. Osteogenic Differentiation by Fraction C

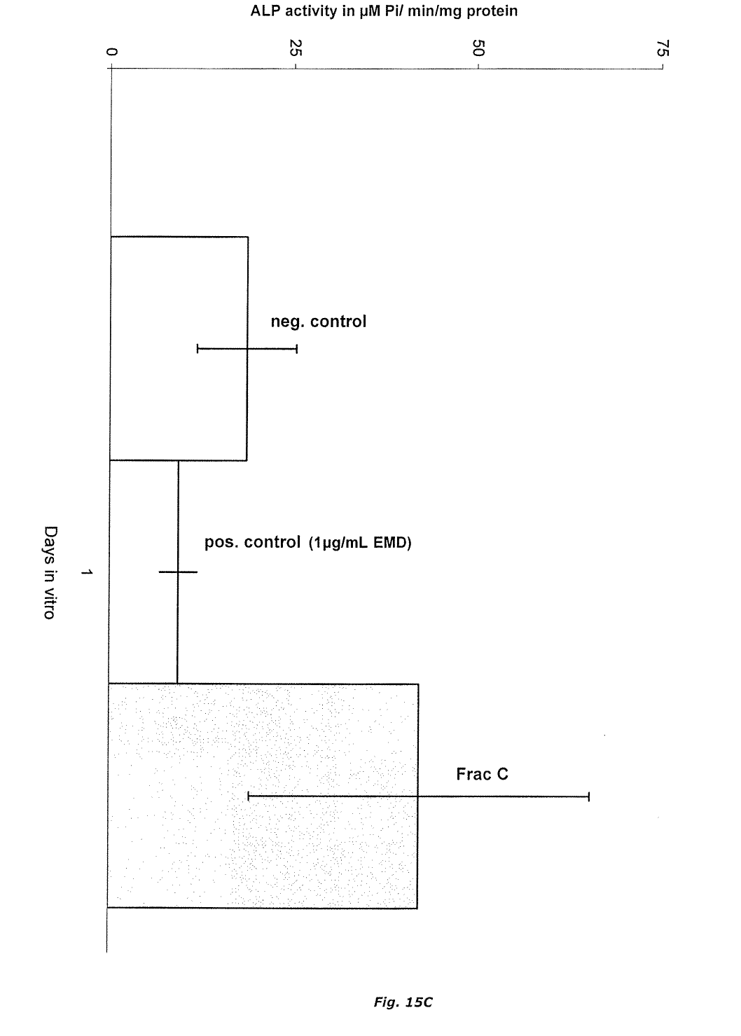

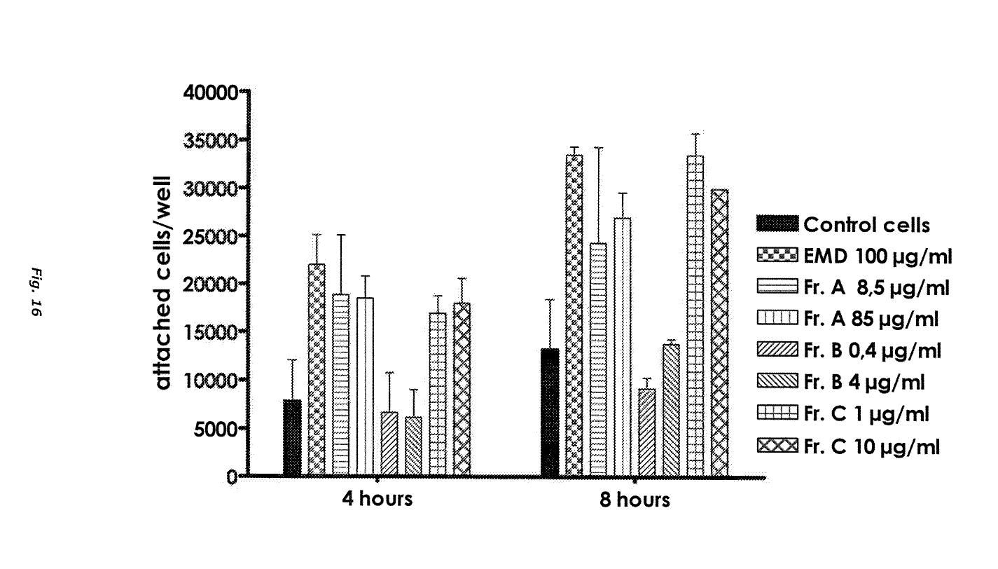

See FIG. 15a, b and c.

Effect of fraction C on osteogenic cells—mineralized connective tissue.

In the present context, both, bone and cementum formation, are included as mineralized tissues.

### Background

The extracellular matrix (ECM) consists mainly of type I collagen. Osteogenic cells synthesize alkaline phosphatase (ALP) playing a key role in mineralization (incorporation of calcium into the ECM). Therefore, ALP is used as an early marker for osteogenic differentiation in vitro. ALP increases within the first days and decreases when mineralization takes place (detection of osteocalcin).

### MG-63

Minimum Essential Medium Eagle (MEM)+10% Fetal calf serum +1% Penicilin-Streptomycin (×100)+1% Non Essential Amino Acid Solution (×100)+1% L-Glutamine (200 mM) Seeding density MG63 cells in passage 4 were seeded with a density of 10,000 cells/cm2 on 96 wells culture plates. Sampling was performed after 24 hours and 7 days in vitro.

### Cell Proliferation and Viability.

To determine the proliferation capacity, we used a colorimetric immunoassay based on the measurement of Bromodeoxyuridine, BrdU incorporation during DNA synthesis. 10 μL of BrdU labelling solution (Roche, Penzberg, Germany) were added to cell culture and cells were incubated for 2 hours at 37° C. The labeling and quantification was carried out as described in the instruction manual. Absorbance was measured with VersaMax Microplate reader at 450 nm with a reference wavelength of 690 nm. Results are reported as optical density (OD).

Furthermore, the proliferation reagent WST-1 test was performed to assess cell proliferation and viability. WST-1 solution was added in a final solution of 1:10 to the monolayer culture and cells were incubated at 37° C. for a further 1 hour. The absorbance of the supernatants was measured spectrophotometrically using VersaMax micro plate reader (Molecular Devices, California, USA) at 420 nm-480 nm with a reference wavelength of 600 nm. Results are reported as optical density (OD).

### Alkaline Phosphatase (ALP) Specific Activity.

For the determination of ALP activity the enzyme activity of the supernatant was assayed spectrophotometrically at 405 nm as the release of p-nitrophenol (Sigma, St. Louis, Mo., USA) from p-nitrophenyl-phosphate over time. MG63 cells were lysed with PBS and 0.05% Triton-X100 on day 1 and 7 after treatment. The ALP was expressed as pM/minute/mg of protein.

### Conditions:

| | |
|---|---|
| 1. Frac C | Fraction of EMD (1 μg/mL) |
| 2. sC1RP − P | synthetic; Peak 1 of Frac C; |
| | phosphorylated (1 μg/mL) |
| 3. sC1RP | synthetic; Peak 1 of Frac C; |
| | non-phosphorylated (1 μg/mL) |
| 4. SC2RP − P | synthetic; Peak 2 of Frac C; |
| | phosphorylated (1 μg/mL) |
| 5. SC2RP | synthetic; Peak 2 of Frac C; |
| | non-phosphorylated (1 μg/mL) |
| 6. sC1RP − P + sC2RP − P | Combination; phosphorylated |
| | (1 μg/mL) |
| 7. sC1RP + SC2RP | Combination; non-phosphorylated |
| | (1 μg/mL) |
| 8. C1RP | Peak 1 of Frac C (1 μg/mL) |
| 9. C2RP | Peak 2 of Frac C (1 μg/mL) |
| 10. C1RP + C2RP | Combination (1 μg/mL) |
| 11. control (100 μg EMD) | Positive control (100 μg/mL) |
| 12. control | Negative control |
| 13. control (1 μg EMD) | Direct positive control (1 μg/mL) |

(s: synthesized; RP: reverse phase; C1: first peak of the chromatogram; C2: second peak of the chromatogram) We have performed 1 experiment with N = 5.

### Medium Change and Sampling:

Medium was changed on day 4 (50%)

Sampling on day 1 (1 day in vitro) and on day 7 (7 days in vitro)

### Results:

### Cell Proliferation Assay:

The results indicate that within the first 24 hours after treatment proliferation capacity was initiated, demonstrated by the increased OD values due to BrdU incorporation. After 7 days proliferative activity of MG63 cells is decreased with confluence and matrix maturation.

Fraction C treatment caused an increase of 10% in proliferative activity after 24 hours. A maximum of 24% increase was detected in cells supplemented with the combination of the synthesized C1RP and C2RP.

### Cell Viability Assay:

With regard to the high proliferation capacity after 24 hours of treatment, MG63 cells demonstrate an increased mitochondrial activity after 7 days. The study showed a stimulation of cell viability and proliferation by all components compared to the negative control (untreated monolayer cultures). Synthesized peptides increase the proliferation of MG63 by 45%, whereby the phosphorylation seems not to enhance the effect. It can be concluded that there is a clear effect of Fraction C components (C1RP, C2RP) on cell proliferation and viability after 7 days in culture, the combination of C1RP+C2RP does increase the viability.

### Alkaline Phosphatase (ALP) Specific Activity:

Alkaline phosphatase is an early marker for osteogenic maturation in vitro. Therefore discussion should mainly focus on results of day 1 rather than day 7 after treatment. ALP activity is typically reduced after 7 days when mineralization takes place. The data indicate that Frac C enhance the ALP activity after 1 day of about 125% compared to the negative control, whereby the single components (C1/C2RP) as well as the combination do not show a significant effect. Furthermore, synthesized peptides demonstrate low ALP activities after 1 day, independent from their phosphorylation status, similar to the negative control. The combination (sC1RP-P+sC2RP-P) of both peptides seems to have a slightly inhibitory effect on ALP activity, which has to be discussed concerning the peptide interaction. Also the positive control (EMD) showed a decrease of ALP activity versus negative control, which has not been demonstrated by supplementation of 100 μg/mL EMD (not shown). Higher concentrations of EMD demonstrate a 1.5 fold increase of ALP activity (versus negative control) after 1 day, and still increasing after 7 days (not shown). This has also been demonstrated by Boyan et al. in former studies, showing almost no effect on the proliferation but on differentiation.

### 8. Attachment of PDL Cells on Different EMD Fractions

Aim: To study attachment of PDL cells on different EMD fractions using XTT assay for assessing of cell number

### Material:

### PDL mix P6, SOP FAM 292

96-well plate for cell suspension
Coating buffer—Carbonate buffer pH 9.6

### XTT kit (Roche)

### Layout:

In 2 96-well plates (4 wells/cond):
1—Control cells
2—EMD 20 ug/well
3—Fraction A 1.7 ug/well
4—Fraction A 17 ug/well
5—Fraction δ 0.08 ug/well
6—Fraction δ 0.8 ug/well
7—Fraction C 0.2 ug/well
8—Fraction C₂ ug/well

### Method:

### 1) Surface Coating

Solution of EMD and fractions are already prepared in bicarbonate buffer for GLP49/37.

200 ul solutions are poured in each well (amount of proteins added to wells is about 5 times lower than in GLP49/37 (200 ul/well instead of 1 ml) as well area is 5.5 times smaller) Incubation overnight at 4 C

The next day, the plates are washed 2 times with 200 ul PBS before adding the cells

### 2) Cell Seeding

One flask of subconfluent PDL mix cells is washed 2 times in PBS and incubated with trypsin/EDTA for 2-3 min. Trypsin is neutralized with medium, cells are spin down and counted. A cell suspension at 50 000 cells/ml is prepared and 200 ul is added in each well (10 000 cells/well)

### 3) XTT Assay

After 4 and 8-hour incubation, floating cells are removed by two washes with 200 ul PBS Wells are refilled with 150 ul normal medium and 75 ul XTT mix is added in each well One extra row containing 150 ul medium and 75 ul XTT mix is added to serve as blank.

Cells are incubated for 2 hours before reading the plate at 480 nm with wavelength reference at 650

### Results:

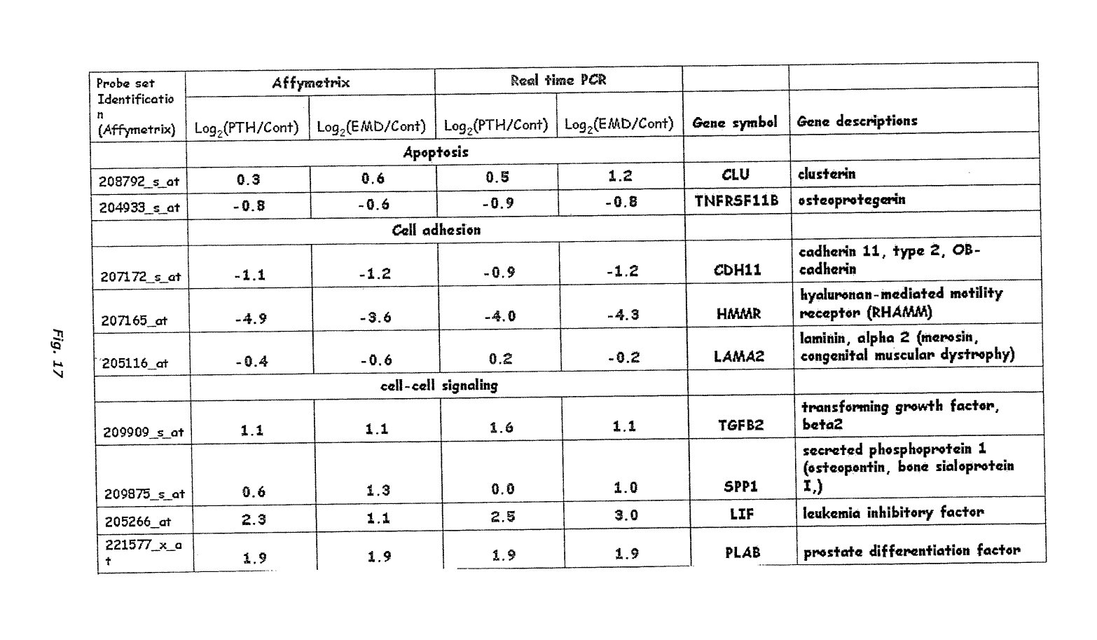

Compare to GLP59/03 PDL mix cells of this experiment attached much quicker to the uncoated plastic. A significant number of cells exhibited spreading in the uncoated condition after 4-hour incubation (In GLP59/03, cells remained round in the uncoated wells after an incubation up to 8 hours) See FIG. 16.

### CONCLUSIONS

The results show a pattern very similar to those obtained in GLP49/37. However the differences between the control and the difference fractions or EMD are not as high (only one condition was significantly different of the control), suggesting that cell number assessment with XTT kit is not very sensitive. Another explanation is that the non-specific attachment of PDL cells is higher in 96-well plate than in 24-well plate reducing the effect (see remark)

### LIST OF REFERENCES

1. Kawase, T. et al. Enamel matrix derivative (EMDOGAIN) rapidly stimulates phosphorylation of the MAP kinase family and nuclear accumulation of smad2 in both oral epithelial and fibroblastic human cells. 3 Periodontal Res 36, 367-76 (2001).
2. Lyngstadaas, S. P., Lundberg, E., Ekdahl, H., Andersson, C. & Gestrelius, S. Autocrine growth factors in human periodontal ligament cells cultured on enamel matrix derivative. J Clin Periodontol 28, 181-8 (2001).
3. Kawase, T., Okuda, K., Yoshie, H. & Burns, D. M. Anti-TGF-beta antibody blocks enamel matrix derivative-induced upregulation of p21WAF1/cipl and prevents its inhibition of human oral epithelial cell proliferation. 3 Periodontal Res 37, 255-62 (2002).
4. Haase, H. R. & Bartold, P. M. Enamel matrix derivative induces matrix synthesis by cultured human periodontal fibroblast cells. 3 Periodontol 72, 341-8 (2001).
5. Van der Pauw, M. T., Van den Bos, T., Everts, V. & Beertsen, W. Enamel matrix-derived protein stimulates attachment of periodontal ligament fibroblasts and enhances alkaline phosphatase activity and transforming growth factor beta1 release of periodontal ligament and gingival fibroblasts. 3 Periodontol 71, 31-43 (2000).
6. Tokiyasu, Y., Takata, T., Saygin, E. & Somerman, M. Enamel factors regulate expression of genes associated with cementoblasts. J Periodontol 71, 1829-39 (2000).
7. Hakki, S. S., Berry, J. E. & Somerman, M. J. The effect of enamel matrix protein derivative on follicle cells in vitro. J Periodontol 72, 679-87 (2001).
8. Brookes, S. J. et al. Amelin extracellular processing and aggregation during rat incisor amelogenesis. Arch Oral Biol 46, 201-8 (2001).
9. Boabaid, F. et al. Leucine-rich amelogenin peptide: a candidate signaling molecule during cementogenesis. J Periodontol 75, 1126-36 (2004).
10. Gestrelius, S., Andersson, C., Lidstrom, D., Hammarstrom, L. & Somerman, M. In vitro studies on periodontal ligament cells and enamel matrix derivative. 3 Clin Periodontol 24, 685-92 (1997).
11. Reseland, J. E. et al. Leptin is expressed in and secreted from primary cultures of human osteoblasts and promotes bone mineralization. J Bone Miner Res 16, 1426-33 (2001).
12. Li, W. et al. X-linked amelogenesis imperfecta may result from decreased formation of tyrosine rich amelogenin peptide (TRAP), Archives of Oral Biology (2003) 48, 177-183.
13. Fincham, A. G. et al., Human Amelogenins: Sequences of “TRAP” Molecules. Calcified Tissue International (1989) 45:243-250.
14. Mumulidu, A. et al., Purification of a 5 kDa component of the enamel matrix derivative. Journal of Chromatography B, 857 (2007) 210-218.
15. WO 03/024479
16. U.S. Pat. No. 4,672,032
17. EP-B-0 337 967
18. EP-B-0 263 086
19. EP-1059934
20. EP-01201915.4
21. WO 01/97834
22. WO 00/53197
23. WO 00/53196
24. WO 02/080994
25. Dayhoff, Schwartz, and Orcutt (1978) Atlas Protein Seq. Struc. 5:345-352
26. Henikoff and Henikoff (1992) Proc Natl Acad Sci USA 89(22):10915-9
27. Simon R J et al., PNAS (1992) 89(20), 9367-9371 and Horwell D C, Trends Biotechnol. (1995) 13(4), 132-134.

1. An active compound of a naturally occurring fraction of Enamel Matrix Derivatives (EMD), comprising at least one of each polypeptide fragments selected from fragments of amelogenin, corresponding to an amino acid sequence being at least 95% identical to the amino acid sequence shown in SEQ. ID. No:1 and 2.

2. An active compound according to claim 1, wherein said two polypeptide fragments of amelogenin, corresponding to an amino acid sequence being at least 95% identical to the amino acid sequence shown in SEQ. ID. No:1 and 2, are dimerized.

3. An active compound according to claim 1, wherein at least one of said polypeptide fragments of amelogenin, corresponding to an amino acid sequence being at least 95% identical to the amino acid sequence shown in SEQ. ID. No:1 and 2, is produced by alternate splicing and/or processing of a natural length amelogenin protein.

4. An active compound according claim 1, wherein at least one of said polypeptide fragments of amelogenin, corresponding to an amino acid sequence being at least 95% identical to the amino acid sequence shown in SEQ. ID. No:1 and 2, is produced by enzymatic and/or chemical cleavage of a natural length amelogenin protein.

5. An active compound according to claim 1 1-6, which is isolated from a porcine, rat, human, and/or mouse enamel matrix protein.

6. An active compound according to claim 1, wherein at least one of said polypeptide fragments of amelogenin, corresponding to an amino acid sequence being at least 95% identical to the amino acid sequence shown in SEQ. ID. No:1 and 2, is produced by synthesis in vitro and/or in vivo.

7. An active compound according to any claim 1, wherein at least one of said polypeptide fragments of amelogenin, corresponding to an amino acid sequence being at least 95% identical to the amino acid sequence shown in SEQ. ID. No:1 and 2, is a purified recombinant polypeptide fragment.

8. An active compound according to claim 1, wherein at least one of said polypeptide fragments of amelogenin, corresponding to an amino acid sequence being at least 95% identical to the amino acid sequence shown in SEQ. ID. No:1 and 2, is synthetically and/or chemically altered.

9. Method of administering an active compound according to claim 1, and/or of an isolated fraction C of EMD for activating and/or regulating activity of periodontal cells.

10. Method of administering an active compound according to claim 1, and/or of an isolated fraction C of EMD for regulating osteoblast differentiation and/or proliferation.

11. Method of administering an active compound according to claim 1, and/or of an isolated fraction C of EMD for regulating mesenchymal stem cell proliferation and/or differentiation.

12. An active compound according to claim 1, and/or an isolated fraction C of EMD for use as a medicament.

13. An active compound according to claim 1, and/or an isolated fraction C of EMD for inducing mineralization in hard tissue.

14. Method of administering an active compound according to claim 1, and/or of an isolated fraction C of EMD for the manufacture of a pharmaceutical composition for inducing mineralization in hard tissue.

15. An active compound according to claim 1, and/or of an isolated fraction C of EMD for inducing bone growth and/or bone regrowth.

16. Method of administering an active compound according to claim 1, and/or of an isolated fraction C of EMD for the manufacture of a pharmaceutical composition for inducing bone growth and/or bone regrowth.

17. Method for inducing bone growth and/or bone regrowth in a mammal, comprising administering an active compound according to claim 1, and/or an isolated fraction C of EMD to a patient in need thereof.


SEQUENCE LISTING (56 sequences)

SEQ ID NO: 1 - protein, 43 aa, Sus scrofa
  MOD_RES (16)..(16): Ser or Glu, wherein Ser is optionally phosphorylated
MPLPPHPGHPGYINFXYEVLTPLKWYQNMIRHPYTSYGYEPMG

SEQ ID NO: 2 - protein, 45 aa, Sus scrofa
  MOD_RES (16)..(16): Ser or Glu, wherein Ser is optionally phosphorylated
MPLPPHPGHPGYINFXYEVLTPLKWYQNMIRHPYTSYGYEPMGGW

SEQ ID NO: 3 - protein, 45 aa, Sus scrofa; identical to SEQ ID NO: 2
  MOD_RES (16)..(16): Ser or Glu, wherein Ser is optionally phosphorylated

SEQ ID NO: 4 - protein, 45 aa, Sus scrofa; identical to SEQ ID NO: 2
  MOD_RES (16)..(16): Ser or Glu, wherein Ser is optionally phosphorylated

SEQ ID NO: 5 - protein, 44 aa, Sus scrofa
  MOD_RES (16)..(16): Ser or Glu, wherein Ser is optionally phosphorylated
MPLPPHPGHPGYINFXYEVLTPLKWYQNMIRHPYTSYGYEPMGG

SEQ ID NO: 6 - protein, 43 aa, Sus scrofa; identical to SEQ ID NO: 1
  MOD_RES (16)..(16): Ser or Glu, wherein Ser is optionally phosphorylated

SEQ ID NO: 7 - protein, 44 aa, Sus scrofa
  MOD_RES (16)..(16): Ser or Glu, wherein Ser is optionally phosphorylated
MPLPPHPGHPGYINFXYEVLTPLKWYQNMIRHPYTSYGYEPGGW

SEQ ID NO: 8 - protein, 43 aa, Sus scrofa
  MOD_RES (16)..(16): Ser or Glu, wherein Ser is optionally phosphorylated
MPLPPHPGHPGYINFXYEVLTPLKWYQNMIRHPYTSYGYEGGW

SEQ ID NO: 9 - protein, 43 aa, Sus scrofa
  MOD_RES (16)..(16): Ser or Glu, wherein Ser is optionally phosphorylated
MPLPPHPGHPGYINFXYEVLTPLKWYQNMIRHPYTSYGYEPGG

SEQ ID NO: 10 - protein, 42 aa, Sus scrofa
  MOD_RES (16)..(16): Ser or Glu, wherein Ser is optionally phosphorylated
MPLPPHPGHPGYINFXYEVLTPLKWYQNMIRHPYTSYGYEGG

SEQ ID NO: 11 - protein, 41 aa, Sus scrofa
  MOD_RES (16)..(16): Ser or Glu, wherein Ser is optionally phosphorylated
MPLPPHPGHPGYINFXYEVLTPLKWYQNMIRHPYTSYGYEG

SEQ ID NO: 12 - protein, 45 aa, Sus scrofa; identical to SEQ ID NO: 2
  MOD_RES (16)..(16): Ser or Glu, wherein Ser is optionally phosphorylated

SEQ ID NO: 13 - protein, 45 aa, Sus scrofa; identical to SEQ ID NO: 2
  MOD_RES (16)..(16): Ser or Glu, wherein Ser is optionally phosphorylated

SEQ ID NO: 14 - protein, 44 aa, Sus scrofa; identical to SEQ ID NO: 5
  MOD_RES (16)..(16): Ser or Glu, wherein Ser is optionally phosphorylated

SEQ ID NO: 15 - protein, 43 aa, Sus scrofa; identical to SEQ ID NO: 1
  MOD_RES (16)..(16): Ser or Glu, wherein Ser is optionally phosphorylated

SEQ ID NO: 16 - protein, 44 aa, Sus scrofa; identical to SEQ ID NO: 7
  MOD_RES (16)..(16): Ser or Glu, wherein Ser is optionally phosphorylated

SEQ ID NO: 17 - protein, 43 aa, Sus scrofa; identical to SEQ ID NO: 8
  MOD_RES (16)..(16): Ser or Glu, wherein Ser is optionally phosphorylated

SEQ ID NO: 18 - protein, 43 aa, Sus scrofa; identical to SEQ ID NO: 9
  MOD_RES (16)..(16): Ser or Glu, wherein Ser is optionally phosphorylated

SEQ ID NO: 19 - protein, 42 aa, Sus scrofa; identical to SEQ ID NO: 10
  MOD_RES (16)..(16): Ser or Glu, wherein Ser is optionally phosphorylated

SEQ ID NO: 20 - protein, 32 aa, Artificial Sequence
  Description of Artificial Sequence Synthetic WBRA001 peptide of amelogenin
MPLPPHPGHPGYINFSYEVLTPLKWYQSIRPP

SEQ ID NO: 21 - protein, 27 aa, Artificial Sequence
  Description of Artificial Sequence Synthetic WBRA005 peptide of amelogenin
HPGHPGYINFSYEVLTPLKWYQSIRPP

SEQ ID NO: 22 - protein, 22 aa, Artificial Sequence
  Description of Artificial Sequence Synthetic WBRA004 peptide of amelogenin
GYINFSYEVLTPLKWYQSIRPP

SEQ ID NO: 23 - protein, 17 aa, Artificial Sequence
  Description of Artificial Sequence Synthetic WBRA003 peptide of amelogenin
SYEVLTPLKWYQSIRPP

SEQ ID NO: 24 - protein, 12 aa, Artificial Sequence
  Description of Artificial Sequence Synthetic WBRA002 peptide of amelogenin
TPLKWYQSIRPP

SEQ ID NO: 25 - protein, 43 aa, Sus scrofa
MPLPPHPGHPGYINFSYEVLTPLKWYQNMIRHPYTSYGYEPMG

SEQ ID NO: 26 - protein, 45 aa, Sus scrofa
MPLPPHPGHPGYINFSYEVLTPLKWYQNMIRHPYTSYGYEPMGGW

SEQ ID NO: 27 - protein, 45 aa, Sus scrofa; identical to SEQ ID NO: 26
  MOD_RES (16)..(16): Optionally phosphorylated

SEQ ID NO: 28 - protein, 44 aa, Sus scrofa
  MOD_RES (16)..(16): Optionally phosphorylated
MPLPPHPGHPGYINFSYEVLTPLKWYQNMIRHPYTSYGYEPMGG

SEQ ID NO: 29 - protein, 43 aa, Sus scrofa; identical to SEQ ID NO: 25
  MOD_RES (16)..(16): Optionally phosphorylated

SEQ ID NO: 30 - protein, 44 aa, Sus scrofa
  MOD_RES (16)..(16): Optionally phosphorylated
MPLPPHPGHPGYINFSYEVLTPLKWYQNMIRHPYTSYGYEPGGW

SEQ ID NO: 31 - protein, 43 aa, Sus scrofa
  MOD_RES (16)..(16): Optionally phosphorylated
MPLPPHPGHPGYINFSYEVLTPLKWYQNMIRHPYTSYGYEGGW

SEQ ID NO: 32 - protein, 43 aa, Sus scrofa
  MOD_RES (16)..(16): Optionally phosphorylated
MPLPPHPGHPGYINFSYEVLTPLKWYQNMIRHPYTSYGYEPGG

SEQ ID NO: 33 - protein, 42 aa, Sus scrofa
  MOD_RES (16)..(16): Optionally phosphorylated
MPLPPHPGHPGYINFSYEVLTPLKWYQNMIRHPYTSYGYEGG

SEQ ID NO: 34 - protein, 41 aa, Sus scrofa
  MOD_RES (16)..(16): Optionally phosphorylated
MPLPPHPGHPGYINFSYEVLTPLKWYQNMIRHPYTSYGYEG

SEQ ID NO: 35 - protein, 45 aa, Sus scrofa; identical to SEQ ID NO: 26
  MOD_RES (16)..(16): Optionally phosphorylated

SEQ ID NO: 36 - protein, 44 aa, Sus scrofa; identical to SEQ ID NO: 28
  MOD_RES (16)..(16): Optionally phosphorylated

SEQ ID NO: 37 - protein, 43 aa, Sus scrofa; identical to SEQ ID NO: 25
  MOD_RES (16)..(16): Optionally phosphorylated

SEQ ID NO: 38 - protein, 44 aa, Sus scrofa; identical to SEQ ID NO: 30
  MOD_RES (16)..(16): Optionally phosphorylated

SEQ ID NO: 39 - protein, 43 aa, Sus scrofa; identical to SEQ ID NO: 31
  MOD_RES (16)..(16): Optionally phosphorylated

SEQ ID NO: 40 - protein, 43 aa, Sus scrofa; identical to SEQ ID NO: 32
  MOD_RES (16)..(16): Optionally phosphorylated

SEQ ID NO: 41 - protein, 42 aa, Sus scrofa; identical to SEQ ID NO: 33
  MOD_RES (16)..(16): Optionally phosphorylated

SEQ ID NO: 42 - protein, 11 aa, Artificial Sequence
  Description of Artificial Sequence Synthetic peptide
SYGYEPMGWLH

SEQ ID NO: 43 - DNA, 20 bp, Artificial Sequence
  Description of Artificial Sequence Synthetic primer
cccagatggagaaagctctg

SEQ ID NO: 44 - DNA, 25 bp, Artificial Sequence
  Description of Artificial Sequence Synthetic primer
gaagcaatatgtgtcatactgggag

SEQ ID NO: 45 - DNA, 24 bp, Artificial Sequence
  Description of Artificial Sequence Synthetic primer
cccacacagacagccactcacctc

SEQ ID NO: 46 - DNA, 24 bp, Artificial Sequence
  Description of Artificial Sequence Synthetic primer
atacctcaaactccaaaagaccag

SEQ ID NO: 47 - DNA, 18 bp, Artificial Sequence
  Description of Artificial Sequence Synthetic primer
cagccgcttcacctacag

SEQ ID NO: 48 - DNA, 25 bp, Artificial Sequence
  Description of Artificial Sequence Synthetic primer
ttttgtattcaatcactgtcttgcc

SEQ ID NO: 49 - DNA, 16 bp, Artificial Sequence
  Description of Artificial Sequence Synthetic primer
gaagcccagcggtgca

SEQ ID NO: 50 - DNA, 19 bp, Artificial Sequence
  Description of Artificial Sequence Synthetic primer
cactacctcgctgccctcc

SEQ ID NO: 51 - DNA, 19 bp, Artificial Sequence
  Description of Artificial Sequence Synthetic primer
gccttcaaggtggtagccc

SEQ ID NO: 52 - DNA, 20 bp, Artificial Sequence
  Description of Artificial Sequence Synthetic primer
cgttacccgccatgacagta

SEQ ID NO: 53 - protein, 4 aa, Unknown
  Description of Unknown DEAD (Asp-Glu-Ala-Asp) box peptide
DEAD

SEQ ID NO: 54 - protein, 39 aa, Sus scrofa
  MOD_RES (16)..(16): Phospho-Ser
MPLPPHPGHPGYINFSYEVLTPLKWYQNMIRHPYTSYGY

SEQ ID NO: 55 - protein, 44 aa, Sus scrofa; identical to SEQ ID NO: 5
  MOD_RES (16)..(16): Phospho-Ser

SEQ ID NO: 56 - protein, 45 aa, Sus scrofa; identical to SEQ ID NO: 26
  MOD_RES (16)..(16): Phospho-Ser